SEQUENCE LISTING (24 sequences)

SEQ ID NO: 1 - protein, 335 aa, Artificial sequence
  Amino acid sequence of recombinant gp120 antigen
MPMGSLQPLATLYLLGMLVASVLATTVLVNVTVTFDMWKNDMVEQMDEAIKTLLDTSLKPCVKLTPLAGATSVITQACPTVSWEPIPIRYCAPPGYAILKCNNKTFNGTGPCTNVSVVTCTHGIRPVVSSQLLLNGSLADEEVVIRSVNFTDNAKTIIVQLNTSVEINCTGAGHCNITRAKWNNTLKQIAEKLREQFGNNKTIIFKQSSGGDPEIVTHWFNCGGEFFYCNSTQLFNSTWFNSTWSTKGSNNTEGSDTITLPCRIKQIGGYAPPVSGVITCSSNITGLLLTRDGGNDNNESEIFRPGGGDMRDNWRSELYKYKVVKLEGSHHHHHH

SEQ ID NO: 2 - protein, 349 aa, Artificial sequence
  Amino acid sequence of recombinant gp120 antigen
MPMGSLQPLATLYLLGMLVASVLATTVLVNVTVTFDWCKNDMVAQMNTAICTLWKTSNKPCVKLTPLCVGAGSCNTSVITQACPTVSFEPIPIRYCAPPGYAILKCNNKTFNGTGPCTNVSVVTCTDGIRPVVSSQLLLNGTLADEEVVIRSCNFTDNAKTIIVQLNTSVEINCTGAGHCNITRAKWNNTLKQIAEKLREQFGNNKTIIFKQSSGGDPEIVTHWFNCGGEFFYCNSTQLFNSTWFNSTWSTKGSNNTEGSDTITLPCRIKQITGMWCTVGKMMYAPPVSGVITCSSNITGLLLTRDGGNDNNESEIFRPGGGDMRDNWRSELYKYKVVKLTGSHHHHHH

SEQ ID NO: 3 - protein, 330 aa, Artificial sequence
  Amino acid sequence of recombinant gp120 antigen
MPMGSLQPLATLYLLGMLVASVLATTVTVNVTVTFDWCADDMVATMNTAICTLWKTSNDPCTKCPTVRFKPVPIRYCAPPGYAILKCNNRDFNGTGPCTNVSVVTCTDGIHPVVSSQLLLNGTLADEKVVIRSCNFSDNAKTIIVQLNTSVEINCTGQGHCNITRAKWNQTLKQIAEKLREQFGNNKTIIFRPSSGGDPEIVTHWFNCGGKFFYCNSTQLFNSTWFNSTWSTKGSNNTEGSDTITLPCRIRSITGMVCTVGKMIYAPPVEGVITCSSNITGLLLTRDGGNDNNESEIFRPGGGDMRDNWRSELYKYRVVRLTGSHHHHHH

SEQ ID NO: 4 - protein, 330 aa, Artificial sequence
  Amino acid sequence of recombinant gp120 antigen
MPMGSLQPLATLYLLGMLVASVLASSVSVNVTQTFSWCDQDMVAKMQQAICNLWQESDTPCNDCPTKAFSPQPIQYCAPNGKAILKCNNENFNGTGPCTNVSVVTCTAGISPVVSSQLLLNGELADETVVIRSCNFNDNAKTIIVQLNTSVEINCTGEGHCNITRAKWNATLKQIAKKLRQQFGNNKTIIFQSSSGGDPEIVTHWFNCGGRFFYCNSTQLFNSTWFNSTWSTKGSNNTEGSDTISLPCRIKSITDMKCSVGKMIYAPPKAGDIKCSSNITGLLLTRDGGNNNNESEIFRPGGGDMRDNWRSELYKYQVVELQGSHHHHHH

SEQ ID NO: 5 - protein, 335 aa, Artificial sequence
  Amino acid sequence of recombinant gp120 antigen
MPMGSLQPLATLYLLGMLVASVLAQKVLVNVTEEFNMWNNNMVELMHQKIASLIKQSLQPCVKLTPLAGATSVITQACPKVDWEPQPIEYCAPDGFAILKCNNSTFNGTGPCTNVSTVRCTHGIRPVVSSQLLLNGSLASSEVVIRSVNFTDNAKTIIVQLNTSVEINCTGDGRCNIARDKWNATLQQIASKLRQQFGSNKTIIFEQSSGGDPEIVTHWFNCGGEFFYCNSTQLFNSTWFNSTWSTEGSNNTEGSDTISLPCRIKQIGGYAPPTRGQIRCSSNITGLLLTRDGGDSSNESEIFRPGGGDMRDNWRSELYKYKVTPIEGSHHHHHH

SEQ ID NO: 6 - protein, 335 aa, Artificial sequence
  Amino acid sequence of recombinant gp120 antigen
MPMGSLQPLATLYLLGMLVASVLAREVLVNVTEQFNMWRNQMVEAMHREIERLERAKLNPCVKLTPLAGATSVITQACPKVQFEPTPITYCAPEGFAILKCNNDTFNGTGPCTNVSTVDCTHGIRPVISSQLLLNGSLAKGEVVIRSVNFTDNAKTIIVQLNTSVEINCTGRGYCNIARKKWNETLEQIASKLRDQFGKNKTIIFSQSSGGDPEIVTHWFNCGGEFFYCNSTQLFNSTWFNSTWSTEGSNNTEGSDTITLPCRIKQIGGYAPPQNGQIRCSSNITGLLLTRDGGPSQNESEIFRPGGGDMRDNWRSELYKYKVKAIEGSHHHHHH

SEQ ID NO: 7 - protein, 335 aa, Artificial sequence
  Amino acid sequence of recombinant gp120 antigen
MPMGSLQPLATLYLLGMLVASVLAKQVLVNTTIHFNMWENSMVQQMHEQIAKLKDQQLEPCVKLTPLAGATSVITQACPVVSWSPEPIKYCAPQGYAILKCNNNTFNGTGPCTNVSEVECTHGIKPVVSSQLLLNGSLANEEVVIRSVNFTDNAKTIIVQLNSSVEINCTGNGHCNITRAKWNQTLKQIAQKLREQFGENKTIIFAQSSGGDPEIVTHWFNCGGEFFYCNSTQLFNSTWFNSTWSTEGSNNTEGSDTIRLPCRIKQIGGYAPPTSGNISCSSNITGLLLTRDGGNRNNNSEIFRPGGGDMRDNWRSELYKYKVVSREGSHHHHHH

SEQ ID NO: 8 - protein, 335 aa, Artificial sequence
  Amino acid sequence of recombinant gp120 antigen
MPMGSLQPLATLYLLGMLVASVLAKQPLQNVTVDFKMWDNDMVDDMHDQIAKEMDEKLSPCVKLTPLAGATSVITQACPKTNWNPVPIKYCAPKGFAILKCNNATFNGTGPCTNVSTVECTHGIRPVVSSQLLLNGSLAETEVVIRSVNFTDNAKTIIVQLNTSVEINCTGDGSCNIARQKWNQTLQQIAEKLRRQFGDNKTIIFRSSSGGDPEIVTHWFNCGGEFFYCNSTRLFNSTWFNSTWSTEGSNNTEGSQTIQLPCRIKQIGGYAPPTQNHIHCSSNITGLLLTRDGGNRNNDSEIFRPGGGDMRDNWRSELYKYKVVKEEGSHHHHHH

SEQ ID NO: 9 - protein, 335 aa, Artificial sequence
  Amino acid sequence of recombinant gp120 antigen
MPMGSLQPLATLYLLGMLVASVLAKTPLPNVTQHFDMWNNNMVEEMHQTIQELLKQQLTPCVKLTPLAGATSVITQACPKRKWDPLPIRYCAPPGFAILKCNNKTFNGTGPCTNVSTVECTHGIRPVVSSQLLLNGSLANTEVVIRSVNFTDNAKTIIVQLNTSVEINCTGNGHCNIAREKWNKTLKQIAKKLREQFGSNKTIIFKSSSGGDPEIVTHWFNCGGEFFYCNSTKLFNSTWFNSTWSTEGSNNTEGSNTIELPCRIKQIGGYAPPTEDNISCSSNITGLLLTRDGGNRDNNSEIFRPGGGDMRDNWRSELYKYKVVQREGSHHHHHH

SEQ ID NO: 10 - DNA, 1008 bp, Artificial sequence
  Neucleic acid sequence of recombinant gp120 antigen
atgcccatgggcagcctgcagcccctggccaccctgtacctgctgggcatgctggtggccagcgtgctggccaccaccgtgctggtgaacgtgaccgtgaccttcgacatgtggaagaacgacatggtggagcagatggacgaggccatcaagaccctgctggacaccagcctgaagccctgcgtgaagctgacccccctggccggcgccaccagcgtgatcacccaggcctgccccaccgtgagctgggagcccatccccatcaggtactgcgccccccccggctacgccatcctgaagtgcaacaacaagaccttcaacggcaccggcccctgcaccaacgtgagcgtggtgacctgcacccacggcatcaggcccgtggtgagcagccagctgctgctgaacggcagcctggccgacgaggaggtggtgatcaggagcgtgaacttcaccgacaacgccaagaccatcatcgtgcagctgaacaccagcgtggagatcaactgcaccggcgccggccactgcaacatcaccagggccaagtggaacaacaccctgaagcagatcgccgagaagctgagggagcagttcggcaacaacaagaccatcatcttcaagcagagcagcggcggcgaccccgagatcgtgacccactggttcaactgcggcggcgagttcttctactgcaacagcacccagctgttcaacagcacctggttcaacagcacctggagcaccaagggcagcaacaacaccgagggcagcgacaccatcaccctgccctgcaggatcaagcagatcggcggctacgccccccccgtgagcggcgtgatcacctgcagcagcaacatcaccggcctgctgctgaccagggacggcggcaacgacaacaacgagagcgagatcttcaggcccggcggcggcgacatgagggacaactggaggagcgagctgtacaagtacaaggtggtgaagctggagggatcccatcatcatcatcatcattag

SEQ ID NO: 11 - DNA, 1050 bp, Artificial sequence
  Neucleic acid sequence of recombinant gp120 antigen
atgcccatgggcagcctgcagcccctggccaccctgtacctgctgggcatgctggtggccagcgtgctggccaccaccgtgctggtgaacgtgaccgtgaccttcgactggtgcaagaacgacatggtggcccagatgaacaccgccatctgcaccctgtggaagaccagcaacaagccctgcgtgaagctgacccccctgtgcgtgggcgccggcagctgcaacaccagcgtgatcacccaggcctgccccaccgtgagcttcgagcccatccccatcaggtactgcgccccccccggctacgccatcctgaagtgcaacaacaagaccttcaacggcaccggcccctgcaccaacgtgagcgtggtgacctgcaccgacggcatcaggcccgtggtgagcagccagctgctgctgaacggcaccctggccgacgaggaggtggtgatcaggagctgcaacttcaccgacaacgccaagaccatcatcgtgcagctgaacaccagcgtggagatcaactgcaccggcgccggccactgcaacatcaccagggccaagtggaacaacaccctgaagcagatcgccgagaagctgagggagcagttcggcaacaacaagaccatcatcttcaagcagagcagcggcggcgaccccgagatcgtgacccactggttcaactgcggcggcgagttcttctactgcaacagcacccagctgttcaacagcacctggttcaacagcacctggagcaccaagggcagcaacaacaccgagggcagcgacaccatcaccctgccctgcaggatcaagcagatcaccggcatgtggtgcaccgtgggcaagatgatgtacgccccccccgtgagcggcgtgatcacctgcagcagcaacatcaccggcctgctgctgaccagggacggcggcaacgacaacaacgagagcgagatcttcaggcccggcggcggcgacatgagggacaactggaggagcgagctgtacaagtacaaggtggtgaagctgaccggatcccatcatcatcatcatcattag

SEQ ID NO: 12 - DNA, 1065 bp, Artificial sequence
  Neucleic acid sequence of recombinant gp120 antigen
atgcctatgggatctctgcagcctctggccacactgtatctgctgggaatgctggtcgcttctgtgctggccacaaccgtgaccgtgaatgtgaccgtgaccttcgattggtgcgccgatgatatggtggctacaatgaacaccgccatctgcaccctgtggaaaaccagcaacgacccctgcaccaagtgtcctaccgtgcggtttaagcccgtgcccatcagatattgtgcccctcctggctatgccatcctgaagtgcaacaaccgggactttaatggcaccggcccttgcacaaatgtgtccgtggtgacctgtacagatggcatccaccctgtggtgtctagtcagctgctgctgaatggcacactggccgatgagaaggtggtgatcagaagctgcaacttcagcgacaacgccaagaccatcatcgtgcagctgaacaccagcgtggagatcaattgtacaggccagggccactgcaatatcacccgggccaagtggaatcagaccctgaagcagatcgccgagaagctgagagagcagttcggcaacaacaagacaatcatcttcaggcctagctctggcggagatcctgagatcgtgacccactggttcaattgcggcggcaagttcttctactgcaacagcacccagctgttcaacagcacctggttcaactctacttggagcaccaagggcagcaacaacaccgagggcagcgataccatcaccctgccctgcaggatcagatctatcaccggcatggtgtgcacagtgggcaagatgatctacgcccctcctgtggaaggcgtgatcacctgcagcagcaacatcacaggcctgctgctgacaagagatggcggcaacgacaacaacgagagcgagatctttagacctggcggcggagacatgagggacaattggcggagcgagctgtacaagtacagagtggtgcggctgaccggatccggcctgaacgacatcttcgaggcccagaagatcgagtggcacgagctggaggtgctgttccagggcccaggccaccaccaccaccaccactga

SEQ ID NO: 13 - DNA, 993 bp, Artificial sequence
  Neucleic acid sequence of recombinant gp120 antigen
atgcctatgggatctctgcagcctctggccacactgtatctgctgggaatgctggtcgcttctgtgctggcctctagcgtgtccgtgaatgtgacccagaccttctcttggtgcgaccaggatatggtggccaaaatgcagcaggccatctgcaatctgtggcaggaaagcgacaccccctgcaatgattgtcccaccaaggcctttagccctcagcctatccagtactgcgcccctaatggcaaggccatcctgaagtgcaacaacgagaacttcaacggcaccggcccttgtacaaatgtgtccgtggtgacctgtacagccggcattagccctgtggtgtctagtcagctgctgctgaatggcgaactggccgatgagacagtggtgatcagaagctgcaacttcaacgacaacgccaagaccatcatcgtgcagctgaacaccagcgtggagatcaattgtacaggcgagggccactgcaatatcacccgggccaagtggaatgccaccctgaagcagatcgccaagaagctgagacagcagttcggcaacaacaagacaattatcttccagtcctcttctggcggagatcctgagatcgtgacccactggttcaattgcggcggcagattcttctactgcaacagcacccagctgttcaacagcacctggttcaactctacttggagcaccaagggcagcaacaacaccgagggcagcgatacaatcagcctgccctgccggatcaagagcatcaccgacatgaagtgcagcgtgggcaagatgatctacgcccctcctaaggccggcgacatcaagtgtagcagcaacatcacaggcctgctgctgacaagagatggcggcaacaataacaacgagagcgagatctttagacctggcggcggagacatgagggacaattggcggagcgagctgtacaagtaccaggtggtggagctgcagggatcccatcatcatcatcatcattag

SEQ ID NO: 14 - DNA, 1008 bp, Artificial sequence
  Neucleic acid sequence of recombinant gp120 antigen
atgcctatgggatctctgcagcctctggccacactgtatctgctgggcatgctggtcgcttcagtgctggctcagaaagtgctggtgaacgtgaccgaggaattcaacatgtggaacaacaacatggtggagctgatgcaccagaagatcgccagcctgatcaaacagagcctgcagccctgtgtgaaactgacacctctggctggcgccacatctgtgatcacccaggcctgtcccaaagtggattgggagccccagcctatcgagtattgcgcccctgatggcttcgccatcctgaagtgcaacaacagcaccttcaatggcaccggcccctgtaccaatgtgtctaccgtgcggtgtacacacggcatcagacctgtggtgtctagccagctgctgctgaatggctctctggccagctctgaggtggtgatcagaagcgtgaacttcaccgacaacgccaagaccatcatcgtgcagctgaacaccagcgtggagatcaattgtaccggcgacggcagatgcaatatcgcccgggacaagtggaatgccacactgcagcagatcgcctccaagctgagacagcagttcggcagcaacaagacaatcatcttcgagcagtcctctggcggagatccagaaatcgtgacccactggttcaactgtggcggcgagttcttctactgcaacagcacccagctgttcaactccacctggttcaatagcacctggtctactgagggaagcaataacaccgagggctccgataccatcagcctgccctgcagaatcaagcagatcggcggctatgctcctcctaccagaggccagatccggtgcagcagcaatatcacaggcctgctgctgacaagagatggcggcgacagcagcaacgagagcgagatctttagacctggcggcggagacatgagagacaattggcggagcgagctgtacaagtacaaagtgacccccatcgagggatcccatcatcatcatcatcattag

SEQ ID NO: 15 - DNA, 1008 bp, Artificial sequence
  Neucleic acid sequence of recombinant gp120 antigen
atgcctatgggatctctgcagcctctggccacactgtatctgctgggcatgctggtcgcttcagtgctggctagagaagtgctggtgaacgtgaccgagcagttcaacatgtggcggaaccagatggtggaggccatgcacagagagatcgagcggctggaaagagccaagctgaacccctgtgtgaaactgacacctctggctggcgccacatctgtgatcacccaggcctgccctaaggtgcagttcgagcccacccctatcacatactgcgcccctgagggctttgccatcctgaagtgcaacaacgacaccttcaatggcaccggcccctgtaccaatgtgtccaccgtggactgtacacacggcatcagacccgtgatctccagccagctgctcctgaatggctctctggccaagggcgaggtggtgatcagaagcgtgaacttcaccgacaacgccaagaccatcatcgtgcagctgaacaccagcgtggagatcaattgcaccggcagaggctactgcaatatcgcccggaagaagtggaacgagacactggaacagatcgccagcaagctgagagatcagttcggcaagaacaagacaatcatcttcagccagtcctctgggggagatccagaaatcgtgacccactggttcaattgtggcggcgagttcttctactgcaacagcacccagctgttcaacagcacctggttcaactccacctggtctacagaaggaagcaataacaccgagggctccgataccatcaccctgccctgcagaatcaagcagatcggcggctatgctcctcctcagaacggccagatccggtgcagcagcaatatcacaggcctgctgctgacaagagatggcggccctagccagaacgagagcgagatctttagacctggcggcggagacatgagagacaattggcggagcgagctgtacaagtacaaagtgaaggccatcgagggatcccatcatcatcatcatcattag

SEQ ID NO: 16 - DNA, 1008 bp, Artificial sequence
  Neucleic acid sequence of recombinant gp120 antigen
atgcctatgggatctctgcagcctctggccacactgtatctgctgggcatgctggtcgcttcagtgctggctaaacaggtgctggtgaacaccaccatccacttcaacatgtgggagaacagcatggtgcagcagatgcacgagcagatcgccaagctgaaggaccagcagctggaaccttgtgtgaagctgacacctctggctggcgccacatctgtgatcacacaggcctgccctgtggtgtcttggagccccgagcctatcaagtattgcgcccctcagggctacgccatcctgaagtgcaacaacaacaccttcaacggcaccggcccctgtacaaatgtgtccgaggtggagtgtacacacggcatcaaaccagtggtctcaagccagctgctgctgaatggcagcctggccaacgaggaagtggtgatcagaagcgtgaacttcaccgacaacgccaagaccatcatcgtgcagctgaacagcagcgtggagatcaattgcaccggcaacggccactgcaatatcacccgggccaagtggaatcagaccctgaagcagattgcccagaagctgagagagcagttcggcgagaacaagacaatcatcttcgcccagagcagtggcggagatcctgagatcgtgacccactggttcaactgtggcggcgagttcttctactgcaactctacccagctctttaattccacatggttcaattccacctggtctacagaaggaagcaataacaccgagggctccgacacaatcagactgccctgccggatcaagcagatcggaggatacgcccctcctaccagcggcaatatcagctgcagcagcaacatcaccggcctgctgctgacaagagatggcggcaaccggaacaacaacagcgagatcttcagacctggcggcggagacatgagagacaattggcggagcgagctgtacaagtacaaggtggtgtcccgggagggatcccatcatcatcatcatcattag

SEQ ID NO: 17 - DNA, 1008 bp, Artificial sequence
  Neucleic acid sequence of recombinant gp120 antigen
atgcctatgggatctctgcagcctctggccacactgtatctgctgggcatgctggtcgcttcagtgctggctaaacagcccctgcagaacgtgaccgtggacttcaagatgtgggacaacgacatggtggacgacatgcacgaccagatcgccaaagagatggacgagaagctgtccccttgtgtgaaactgacacctctggctggcgccacatctgtgatcacccaggcctgccccaagaccaattggaaccccgtgcccatcaagtactgcgcccccaagggctttgccatcctgaagtgcaacaacgccacctttaatggcaccggcccctgcacaaatgtgtccaccgtggagtgtacacacggcatcagacctgtggtgtctagccagctgctgctgaatggctctctggccgagacagaggtggtgatcagaagcgtgaacttcaccgacaacgccaagaccatcatcgtgcagctgaacaccagcgtggagatcaattgtaccggcgacggcagctgtaatatcgcccggcagaagtggaatcagaccctgcagcagatcgccgagaagctgagaaggcagttcggcgacaacaagacaatcatcttcagaagcagctcaggaggagatcctgagatcgtcacccactggttcaactgtggcggcgagttcttctactgcaacagcacccggctgttcaacagcacctggttcaattccacctggtccaccgagggcagcaataatacagagggcagccagaccattcagctcccttgtaggatcaagcagatcggcggctatgcccctcctacccagaaccacatccactgcagcagcaatatcaccggcctgctgctgacaagagatggcggcaaccggaacaacgacagcgagatctttagacctggcggcggagacatgagagacaattggcggagcgagctgtacaagtacaaggtggtgaaagaggaaggatcccatcatcatcatcatcattag

SEQ ID NO: 18 - DNA, 1008 bp, Artificial sequence
  Neucleic acid sequence of recombinant gp120 antigen
atgcctatgggatctctgcagcctctggccacactgtatctgctgggcatgctggtcgctagcgtgctggctaagacccctctgcctaacgtgacccagcacttcgacatgtggaacaacaacatggtggaggaaatgcaccagaccatccaggaactgctgaaacagcagctcaccccttgtgtgaaactgacacctctggctggcgccacatctgtgatcacccaggcctgccccaaaagaaaatgggaccccctgcccatcagatattgcgcccctcctggctttgccatcctgaagtgcaacaacaagaccttcaatggaaccggaccctgtacaaatgtgtccaccgtggagtgtacacacggcatcagacctgtggtgtctagccagctgctcctgaatggcagcctggccaataccgaggtggtgatcagaagcgtgaacttcaccgacaacgccaagaccatcatcgtgcagctgaacaccagcgtggagatcaattgcaccggcaacggccactgtaatatcgcccgggagaagtggaataagaccctgaagcagatcgccaagaagctgagagagcagttcggcagcaacaagacaatcatcttcaagagcagcagcggcggagatccagaaatcgtgacccactggttcaactgtggcggcgagttcttctactgcaacagcaccaagctgttcaacagcacctggttcaattccacctggtccacagagggaagcaataacaccgagggctccaacacaatcgagctgccctgcagaatcaagcagatcggcggctatgcccctcctaccgaggacaacatcagctgcagcagcaacatcacaggcctgctgctgacaagagatggcggcaatagagataacaacagcgagatcttcagacctggaggaggggacatgagagacaattggcggagcgagctgtacaagtacaaggtggtgcagcgggagggatcccatcatcatcatcatcattag

SEQ ID NO: 19 - protein, 317 aa, Artificial Sequence
  Amino acid sequence of recombinant gp120 antigen
QLAVLNVTQSFDAYNHTMVQEAIDNVWQLWETTIKPCVKLSPICIGAGHCNTSVVQESCDTHYWDAVRIRYCAPPGYAIMRCNNKTFNGTMPCSNVSVSSCTRMIEPVVSTQLLLNGSEARTYVYWRSVNFTENATIIIVNLNTSVTIKCRGAGWCNISGAQWNNTLKEIAQTLVKHPRNNKTIIFKQSSGGDPEIVTHSFNCGGEFFYCNSTWLFNSTWFNSTYTSDGTNNTKEQHRIYLPCRIRQIVTTWHRVGKNVYLPPREGDLTCNSNITGLLLTRDGGDGNNESITFRPGGGDMRDNWRSDLYDYQLVEIT

SEQ ID NO: 20 - protein, 316 aa, Artificial Sequence
  Amino acid sequence of recombinant gp120 antigen
EVVLVNVTENFNWCKNDMVEQMHEDICSLWDQSLKPCVKLTPLCVGAGSCNTSVITQACPKVSFEPIPIHYCAPAGFAILKCNNKTFNGTGPCTNVSTVCTHGIRPVVSSQLLLNGSLAEEEVVIRSCNFTDNAKTIIVQLNTSVEINCTGAGHCNISRAKWNNTLKQIASKLREQFGNNKTIIFKQSSGGDPEIVTHWFNCGGEFFYCNSTQLFNSTWFNSTWSTEGSNNTEGSDTITLPCRIKQIINMWCKVGKMMYAPPISGQIRCSSNITGLLLTRDGGNSNNESEIFRPGGGDMRDNWRSELYKYKVVKIE

SEQ ID NO: 21 - protein, 26 aa, Artificial Sequence
  Synthetic peptide sequence
GYIPEAPRDGQAYVRKDGEWVLLSTF

SEQ ID NO: 22 - protein, 18 aa, Artificial Sequence
  Synthetic peptide sequence
LIVLGGVAGLLLFIGLGI

SEQ ID NO: 23 - protein, 6 aa, Artificial Sequence
  Synthetic hexapeptide.
NTRGRR

SEQ ID NO: 24 - protein, 16 aa, Artificial Sequence
  Synthetic peptide
VKLTPLAGATSVITQA

(19) United States
(12) Patent Application Publication
(10) Pub. No.: US 2012/0034254 A1
(43) Pub. Date: Feb. 9, 2012

(54) ANTIGENIC CLOAKING AND ITS USE

(76) Inventors: Peter D. Kwong, Washington, DC (US); Gary J. Nabel, Washington, DC (US); George Shaw, Birmingham, AL (US); William Schief, Seattle, WA (US); Zhi-yong Yang, Potomac, MD (US); Tongqing Zhou, Boyds, MD (US); Lei Chen, Rockville, MD (US); Young Do Kwon, Rockville, MD (US)

(21) Appl. No.: 12/866,723

(22) PCT Filed: Feb. 6, 2009
(86) PCT No.: PCT/US2009/033457

§ 371 Date: Aug. 6, 2010

Related U.S. Application Data
(60) Provisional application No. 61/065,114, filed on Feb. 7, 2008.
(60) Provisional application No. 61/065,896, filed on Feb. 14, 2008.

(51) Int. Cl. A61K 39/21; C07K 14/195; C07K 14/37; C07K 14/005; A61P 31/18; A61K 39/00; C07H 21/00; G01N 33/566; A61P 37/04; C07K 14/16; C07H 21/04

(52) U.S. Cl. 424/188.1; 530/395; 530/350; 536/23.72; 424/184.1; 536/23.1; 424/208.1; 435/7.92

(57) ABSTRACT

Disclosed are antigens that include a target epitope that is defined by atomic coordinates of those amino acids of the antigen that contact an antibody of interest that specifically binds the antigen. The disclosed antigens have between about 10% and about 90% of surface exposed amino acid residues located exterior of the target epitope substituted as compared to a wild-type antigen and less than about 10% of the non-surface exposed amino acid residues substituted as compared to a wild-type antigen. Also disclosed are nucleic acids encoding these antigens and methods of producing these antigens. Methods for generating an immune response in a subject are also disclosed. In some embodiments, the method is a method for treating or preventing a human immunodeficiency type 1 (HIV-1) infection in a subject.

### CROSS REFERENCE TO RELATED APPLICATION

This application claims the benefit of U.S. Provisional Application No. 61/065,114, filed Feb. 7, 2008, and U.S. Provisional Application No. 61/065,896, filed Feb. 14, 2008. Both of these provisional applications are incorporated by reference herein in their entirety.

### STATEMENT OF JOINT RESEARCH

The research work described here was performed under a Cooperative Research and Development Agreement (CRADA) between the U.S. Government (NIAID CRADA AI-0156 (WKMS Tracking Code 2006-0370)) and International AIDS Vaccine Initiative (IAVI) entitled “The Rational Design of HIV Envelope Glycoprotein Variants for Structural and Immunological Analysis.”

### FIELD

The present disclosure relates to immunogenic polypeptides, and specifically to focusing the immune response to target epitopes on pathogens by antigenic cloaking.

### BACKGROUND

Over the past century the development of agents to combat infections, such as viral infections, fungal infections, bacterial infections and the like, has vastly increased the average lifespan throughout the world. However, pathogens are increasingly developing ways to avoid or circumvent existing therapeutic agents. For example, the widespread use of traditional antibiotics, such as penicillin and related compounds, has resulted in the development of bacteria that are resistant to these traditional antibiotics. This antibiotic resistance is exemplified by the rise of methicillin resistant Staphylococcus aureus (MRSA). Similarly, viral pathogens, such as human immunodeficiency virus (HIV), are able to acquire resistance to antivirals within a few replication cycles.

To combat the ever-changing landscape of pathogens and emerging resistance to the current therapies, the standard course of action for pharmaceutical companies is to develop an ever-increasing array of small molecule therapeutic agents. As an alternative, vaccines have been developed which stimulate the body to fight an infection by eliciting antibody responses to the target pathogen(s). In some examples, these vaccines are polypeptide epitopes that induce an immune response to pathogens and can be referred to as immunogens. These immunogens can be introduced into a subject where they can elicit an antibody response to specific epitopes of the pathogen. For example, immunogens derived from the envelope protein of HIV have been used to produce an antibody response.

An enveloped virus, HIV-1 hides from humoral recognition behind a protective lipid bilayer. An available viral target for neutralizing antibodies is the envelope spike. The major envelope protein of HIV-1 is a glycoprotein of approximately 160 kD (gp160). During infection proteases of the host cell cleave gp160 into gp120 and gp41. The gp41 is an integral membrane protein, while gp120 protrudes from the mature virus. Together gp120 and gp41 make up the HIV envelope spike.

The mature gp120 glycoprotein is approximately 470-490 amino acids long depending on the HIV strain of origin. N-linked glycosylation at approximately 20-25 sites makes up nearly half of the mass of the molecule. Sequence analysis shows that the polypeptide is composed of five conserved regions (C1-C5) and five regions of high variability (V1-V5).

It is believed that immunization with effectively immunogenic HIV gp120 envelope glycoprotein can elicit a neutralizing response directed against gp120, and thus HIV. The need exists for immunogens that are capable of eliciting an immunogenic response in a suitable subject. In order to be effective, the antibodies raised must be capable of neutralizing a broad range of HIV strains and subtypes.

Thus, there is a need for immunogens that can be used to elicit an immune response to pathogens, such as HIV.

### SUMMARY

Antigens are disclosed that have been designed to include a target epitope that is defined by atomic coordinates of those amino acids of the antigen that contact an antibody of interest that specifically binds the antigen. These antigens have between about 10% and about 90% of surface exposed amino acid residues located exterior of the target epitope substituted as compared to a wild-type antigen. These antigens have less than about 10% of the non-surface exposed amino acid residues substituted as compared to a wild-type antigen. The amino acid substitutions alter antigenicity of the antigen in vivo as compared to the wild-type antigen, but do not introduce additional glycosylation sites as compared to the wild-type antigen and do not significantly alter the binding of the antigen to the antibody of interest. In some embodiments, the amino acid substitutions result in the antigen not being bound by antibodies in a polyclonal serum that specifically bind surface exposed amino acid residues of the wild-type antigen located exterior of the target epitope. The antigens can be from a pathogen, such as a viral, bacterial or fungal pathogen. Nucleic acid molecules encoding the disclosed antigens are also disclosed. In some embodiments, the antigen is a human immunodeficiency virus (HIV)-1 antigen, such as a gp120, gp41, gp140 or gp160 or an immunogenic fragment thereof.

Also disclosed are methods of producing the disclosed antigens. In some examples, an antigen is produced by obtaining the atomic coordinates of a wild-type antigen, wherein a monoclonal antibody specifically binds the wild-type antigen and amino acids of the wild-type antigen that contact the antibody have been identified. The amino acids of the wild-type antigen that contact the monoclonal antibody are selected as the target epitope and at least one surface exposed amino acid residue located exterior to the target epitope of the wild-type antigen is selectively mutated. The affinity of the monoclonal antibody for the antigen is not significantly altered by the selective mutation of the surface exposed amino acid residues located exterior to the target epitope. In some examples, the antigen is gp120 and the antigen is selectively mutated by mutating the amino acid residues exterior to the target epitope to homologous residues from gp120 from SIV or HIV2.

Methods for generating an immune response in a subject are disclosed. The methods include administering to the subject a therapeutically effective amount of the pharmaceutical composition that includes a disclosed antigen. In some embodiments, the methods are methods for treating or preventing a human immunodeficiency type 1 (HIV-1) infection in a subject.

### BRIEF DESCRIPTION OF THE DRAWINGS

FIG. 1 is a flow diagram showing an exemplary procedure for computational design of a cloaked antigen.

FIG. 2 is a flow diagram showing an exemplary procedure for computational design of an antigenically-cloaked gp120 antigen in which surface exposed residues of HIV-1 gp120 that are not in contact with the neutralizing antibody b12 are replaced with the residues present in the homologous position of HIV-2 or SIV.

FIG. 3 is a Table and a ribbon diagram showing the amino acid positions of the gp120 polypeptide that contact the neutralizing antibody b12. The b12 antibody binding site is made from multiple non-contiguous regions of the gp120 amino acid sequence.

FIG. 4 is a set of surface representations of gp120 showing the location of the b12 antibody binding site and possible sites on gp120 that could be mutated to antigenically-cloak gp120, for example to focus the antigenicity of gp120 to the b12 binding site.

FIG. 5 is a set of surface representations and a stick model of the atomic coordinates of an antigenically-cloaked gp120 in which surface residues of gp120 have been systematically mutated to the homologous residues from SIVmac239 in a WT Core context (the bridging-sheet was removed and a modified V3 was added). The sites of mutation are shown in black. The b12 binding site is circled.

FIG. 6 is a sequence alignment of the New_SIVmac239_cloaked_core amino acid sequence (SEQ ID NO: 19) and the HXB2_core_{—}8b amino acid sequence (SEQ ID NO: 20).

FIG. 7 is a set of surface representations and a ribbon diagram of the New 2NXY_{—}11b_F105_{—}1 atomic coordinates and a Table showing the location of the F105 antibody contact surface (the CD4 binding loop). The sites of introduced mutations used to cloak the antigen and focus antigenicity to the F105 binding site are shown in black.

FIG. 8 is a set of traces showing the binding of the gp120 antigens WT Hxbc2 core, full length Hxbc2 and 2nxy_{—}11b_f105_{—}1 to the antibody b12 at a single concentration.

FIG. 9 is a set of traces showing the binding of 2nxy_{—}11b_f105_{—}1 to the b12 antibody, CD4 and F105.

FIG. 10 is a set of traces showing the binding of the gp120 antigens WT Hxbc2 core, full length Hxbc2 and 2nxy_{—}11b_f105_{—}1 to CD4 at a single concentration.

FIG. 11 is three sets of kinetics traces used to determine the kinetics of WT Hxbc2 core, full length Hxbc2 and 2nxy_{—}11b_f105_{—}1 to CD4. The on rate (Kₒₙ), the off rate (K_{off}) and the dissociation constant (K_{d}) are shown.

FIG. 12 is three sets of kinetics traces used to determine the kinetics of WT Hxbc2 core, full length Hxbc2 and 2nxy_{—}11b_f105_{—}1 to the b12 antibody. The on rate (Kon), the off rate (Koff) and the dissociation constant (KD) are shown.

FIG. 13 is a set of graphs showing the binding of antigenically-cloaked gp120 antigens to CD4, the 2G12 antibody, the b12 antibody, the b13 antibody and subject sera (normal, Pt. 1 and 1642) and human IgG. The presence of the D368R mutation abolishes binding to all but the 2G12 antibody.

FIG. 14 is a block diagram of a computer system that can be used to implement aspects of the present disclosure.

FIG. 15 is a diagram of a distributed computing environment in which aspects of the present disclosure can be implemented.

FIG. 16 is a set of surface representations of the atomic coordinates of Core/8b gp120 and the antigenically-cloaked gp120 antigens 2NXY_{—}11b_{—}1, SIV-8b-sg-11b and SIV_{—}8b_{—}11b_{—}2a, 2NXY-11b-comp-2g_{—}0017, and 2NXY-11b-comp_{—}6e_{—}0007. The sites of mutations introduced to cloak the antigen are shown in black.

FIG. 17 is a set of surface representations of the atomic coordinates of the antigenically-cloaked gp120 antigens 2NXY-11b-redes-8_{—}0105, 2NXY-11c-25_{—}018 and 2NXY-polar1pt5_{—}0177. The sites of mutations introduced to cloak the antigens are shown in black.

FIG. 18 is a Table showing the serum dilutions needed to achieve 50% neutralization of the indicated viral particle. Sera was obtained from rabbits at the indicated time points and used in neutralization trials. A larger number indicates that the sera has a high level of neutralizing antibodies. The antigens used in the individual trails is given in the fourth column. The immunization schedule is given in the third column.

FIG. 19 is a Table showing the serum dilutions needed to achieve 80% neutralization of the indicated viral particle. Sera was obtained from rabbits at the indicated time points and used in neutralization trials. A larger number indicates that the sera has a high level of neutralizing antibodies. The antigens used in the individual trails is given in the fourth column. The immunization schedule is given in the third column.

FIG. 20 is a Table showing the serum dilutions needed to achieve 50% neutralization of the indicated viral particle. Sera was obtained from rabbits at the indicated time points and used in neutralization trials. A larger number indicates that the sera has a high level of neutralizing antibodies. The immunization schedule is given in FIG. 22.

FIG. 21 is a Table showing the serum dilutions needed to achieve 80% neutralization of the indicated viral particle. Sera was obtained from rabbits at the indicated time points and used in neutralization trials. A larger number indicates that the sera has a high level of neutralizing antibodies. The immunization schedule is given in FIG. 22.

FIG. 22 is a Table showing the immunization schedule for neutralization trials shown in FIGS. 20 and 21.

FIG. 23 is a Table showing tabulating the data for listed gp120 antigens. Column one is the antigen name, column two is the percentage of cloaking, column three indicates whether the antigen has the β20/21 region deleted, column four indicates whether the construct has the D368R mutation, column five shows the number of introduced glycan sites, column six indicates the protein yield per liter of culture, column seven indicates the relative degree to which the construct binds CD4, column eight indicates the relative degree to which the construct binds IgG1b12, column nine indicates the relative degree to which the construct binds Pt#1 sera, column ten indicates the relative degree to which the construct binds B7B5, column eleven indicates the relative degree to which the construct binds 2G12.

FIG. 24 is a graph showing antigenicity profile of the SIV_{—}8b_{—}11b_{—}2a antigen to selected antibodies.

FIG. 25 is two graphs showing the binding of SIV_{—}8b_{—}11b_{—}2a antigen to antibodies present in rabbit polyclonal sera.

FIG. 26 is two graphs showing the binding of SIV_{—}8b_{—}11b_{—}2a antigen to antibodies present in rabbit polyclonal sera can be competed by the b12 antibody.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NOs: 1-9, 19 and 20 are the amino acid sequences of exemplary gp120 antigens.

SEQ ID NOs: 10-18 are the nucleic acid sequences encoding exemplary gp120 antigens.

SEQ ID NO: 21 is the amino acid sequence of an exemplary fibritin foldon.

SEQ ID NO: 22 is the amino acid sequence of an exemplary hCD transmembrane domain.

SEQ ID NO: 23 is the amino acid sequence of an exemplary hexapeptide.

SEQ ID NO: 24 is the amino acid sequence of an a peptide.

### DETAILED DESCRIPTION

### I. Summary of Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen”

As used herein, the term “comprises” means “includes.” Thus, “comprising an antigen” means “including an antigen” without excluding other elements.

It is further to be understood that all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the invention, the following explanations of terms are provided:

2F5: A broadly neutralizing antibody that specifically binds human immunodeficiency virus type 1 (HIV-1) antigen.

2G12: A broadly neutralizing antibody that specifically binds HIV-1 antigen.

4E10: A broadly neutralizing antibody that specifically binds HIV-1 antigen.

Adjuvant: A vehicle used to enhance antigenicity. Adjuvants include a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages) Immunstimulatory oligonucleotides (such as those including a CpG motif) can also be used as adjuvants (for example see U.S. Pat. No. 6,194,388; U.S. Pat. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199). Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L and 41 BBL. Adjuvants can be used in combination with the disclosed antigens.

Administration: The introduction of a composition into a subject by a chosen route. For example, if the chosen route is intravenous, the composition (such as a disclosed antigen) is administered by introducing the composition into a vein of the subject.

Alanine-scanning mutagenesis: A systematic mutational screen where the amino acids of a protein of interest, such as an antigen, for example gp120, are mutated to alanine to determine the effect of the alanine substitution at that position. Alanine-scanning mutagenesis can be used to locate a target epitope, for example by determining if a specific alanine mutation inhibits the specific binding of an antibody of interest to an antigen.

Amino acid substitutions: The replacement of one amino acid in an antigen with a different amino acid. In some examples, an amino acid in an antigen is substituted with an amino acid from a homologous antigen.

Amplification: A technique that increases the number of copies of a nucleic acid molecule (such as an RNA or DNA). An example of amplification is the polymerase chain reaction, in which a biological sample is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Animal: A living multi-cellular vertebrate or invertebrate organism, a category that includes, for example, mammals. The term mammal includes both human and non-human mammals. Similarly, the term “subject” includes both human and veterinary subjects, such as non-human primates. Thus, administration to a subject can include administration to a human subject. Particular examples of veterinary subjects include domesticated animals (such as cats and dogs), livestock (for example, cattle, horses, pigs, sheep, and goats), laboratory animals (for example, mice, rabbits, rats, gerbils, guinea pigs, and non-human primates).

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, antigens include peptides derived from a pathogen of interest. Exemplary pathogens include bacteria, fungi, viruses and parasites. In specific examples, an antigen is derived from HIV, such as a gp120 polypeptide or antigenic fragment thereof, such as a gp120 outer domain.

A “target epitope” is a specific epitope on an antigen that specifically binds a antibody of interest, such as a monoclonal antibody. In some examples, a target epitope includes the amino acid residues that contact the antibody of interest, such that the target epitope can be selected by the amino acid residues determined to be in contact with the antibody of interest.

Antigenically-cloaked immunogen or Antigenically-cloaked Antigen: A polypeptide immunogen derived from a wild-type antigen in which amino acid residues outside or exterior to a target epitope are mutated in a systematic way to focus the immunogenicity of the antigen to the selected target epitope.

Antibody: A polypeptide substantially encoded by an immunoglobulin gene or immunoglobulin genes, or fragments thereof, which specifically binds and recognizes an analyte (such as an antigen or immunogen) such as gp120 or an antigenic fragment of gp120 or an antigenically-cloaked gp120 antigen. Immunoglobulin genes include the kappa, lambda, alpha, gamma, delta, epsilon and mu constant region genes, as well as the myriad immunoglobulin variable region genes.

Antibodies exist, for example as intact immunoglobulins and as a number of well characterized fragments produced by digestion with various peptidases. For instance, Fabs, Fvs, and single-chain Fvs (SCFvs) that bind to gp120 or fragments of gp120 would be gp120-specific binding agents. This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies (such as bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. The term “antibody,” as used herein, also includes antibody fragments either produced by the modification of whole antibodies or those synthesized de novo using recombinant DNA methodologies.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for binding to an epitope of an antigen.

The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Light chain CDRs are sometimes referred to as CDR L1, CDR L2, and CDR L3. Heavy chain CDRs are sometimes referred to as CDR H1, CDR H2, and CDR H3.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Humanized immunoglobulins can be constructed by means of genetic engineering (for example, see U.S. Pat. No. 5,585,089).

Atomic Coordinates or Structure coordinates: Mathematical coordinates derived from mathematical equations related to the patterns obtained on diffraction of a monochromatic beam of X-rays by the atoms (scattering centers) such as an antigen, or an antigen in complex with an antibody. In some examples that antigen can be gp120, or a gp120: antibody complex, or combinations thereof in a crystal in crystal form. The diffraction data are used to calculate an electron density map of the repeating unit of the crystal. The electron density maps are used to establish the positions of the individual atoms within the unit cell of the crystal. In one example, the term “structure coordinates” refers to Cartesian coordinates derived from mathematical equations related to the patterns obtained on diffraction of a monochromatic beam of X-rays, such as by the atoms of a gp120 in crystal form.

Those of ordinary skill in the art understand that a set of structure coordinates determined by X-ray crystallography is not without standard error. For the purpose of this disclosure, any set of structure coordinates that have a root mean square deviation of protein backbone atoms (N, Cα, C and 0) of less than about 1.0 Angstroms when superimposed, such as about 0.75, or about 0.5, or about 0.25 Angstroms, using backbone atoms, shall (in the absence of an explicit statement to the contrary) be considered identical.

Bacterial pathogen: A bacteria that causes disease (pathogenic bacteria). Examples of pathogenic bacteria for which a disclosed antigen can be produced to elicit an immune response in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. Coli, enteroinvasive E. Coli, enteropathogenic E. Coli, enterohemorrhagic E. Coli, enteroaggregative E. Coli and uropathogenic E. Coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (such as Mycobacterium leprae, Mycobacterium tuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgicao and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaminogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio fumisii), Yersinia sp. (such as Yersinia enterocolitica, Yersinia pestis, and Yersinia pseudotuberculosis) and Xanthomonas maltophilia among others.

CD4: Cluster of differentiation factor 4 polypeptide, a T-cell surface protein that mediates interaction with the MHC class II molecule. CD4 also serves as the primary receptor site for HIV on T-cells during HIV-1 infection.

The known sequence of the CD4 precursor has a hydrophobic signal peptide, an extracelluar region of approximately 370 amino acids, a highly hydrophobic stretch with significant identity to the membrane-spanning domain of the class II MHC beta chain, and a highly charged intracellular sequence of 40 residues (Maddon, Cell 42:93, 1985).

The term “CD4” includes polypeptide molecules that are derived from CD4 include fragments of CD4, generated either by chemical (for example enzymatic) digestion or genetic engineering means. Such a fragment may be one or more entire CD4 protein domains. The extracellular domain of CD4 consists of four contiguous immunoglobulin-like regions (D1, D2, D3, and D4, see Sakihama et al., Proc. Natl. Acad. Sci. 92:6444, 1995; U.S. Pat. No. 6,117,655), and amino acids 1 to 183 have been shown to be involved in gp120 binding.

b12 antibody: A specific broadly neutralizing antibody against human immunodeficiency virus type 1 (HIV-1). The epitope recognized by b12 overlaps the CD4 receptor-binding site (CD4BS) on gp120, see Zwick et al., J. of Virology 77: 5863-5870, 2003, herein incorporated by reference.

CD4BS antibodies: Antibodies that bind to or substantially overlap the CD4 binding surface of a gp120 polypeptide. The antibodies interfere with or prevent CD4 from binding to a gp120 polypeptide.

CD4i antibodies: Antibodies that bind to a conformation of gp120 induced by CD4 binding.

CD8: Cluster of differentiation factor 8, a T cell surface protein that mediates interaction with the MHC Class 1 molecule. Cells that express CD8 are often cytotoxic T cells.

Contacting: Placement in direct physical association; includes both in solid and liquid form. Contacting includes contact between one molecule and another molecule, for example the amino acid on the surface of one polypeptide, such as a antigen, that contact another polypeptide, such as an antibody.

Computer readable media: Any medium or media, which can be read and accessed directly by a computer, so that the media is suitable for use in a computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

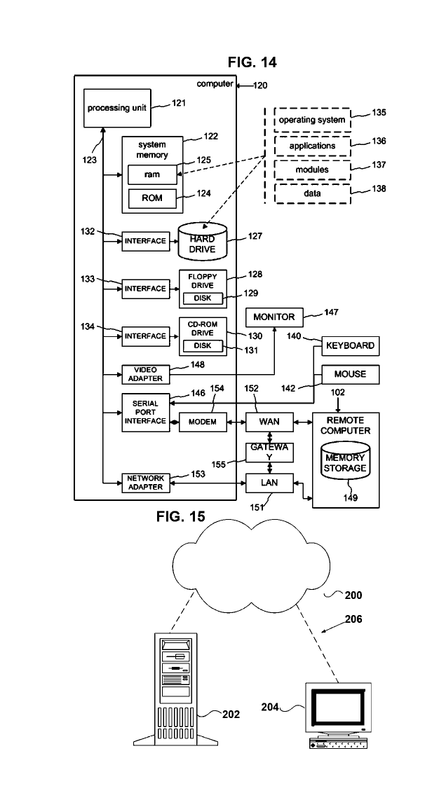

Computer system: Hardware that can be used to analyze atomic coordinate data and/or design an antigen using atomic coordinate data. The minimum hardware of a computer-based system typically comprises a central processing unit (CPU), an input device, for example a mouse, keyboard, and the like, an output device, and a data storage device. Desirably a monitor is provided to visualize structure data. The data storage device may be RAM or other means for accessing computer readable. Examples of such systems are microcomputer workstations available from Silicon Graphics Incorporated and Sun Microsystems running Unix based Windows NT or IBM OS/2 operating systems. An exemplary computer system that can be used with the methods of this disclosure is depicted in FIG. 14.

Degenerate variant and conservative variant: A polynucleotide encoding a polypeptide or an antibody that includes a sequence that is degenerate as a result of the genetic code. For example, a polynucleotide encoding a disclosed antigen or an antibody that specifically binds a disclosed antigen includes a sequence that is degenerate as a result of the genetic code. There are 20 natural amino acids, most of which are specified by more than one codon. Therefore, all degenerate nucleotide sequences are included as long as the amino acid sequence of the antigen or antibody that binds the antigen encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation.

One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

One of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

Expression: Translation of a nucleic acid into a protein. Proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

Foldon domain: An amino acid sequence that naturally forms a trimeric structure. In some examples, a foldon domain can be included in the amino acid sequence of a disclosed antigen so that the antigen will form a trimer. In one example, a foldon domain is the T4 foldon domain.

Fungal pathogen: A fungus that causes disease. Examples of fungal pathogens for which antigens can be produced to elicit an immune response in accordance with the disclosed methods include without limitation Trichophyton rubrum, T. mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum) among others.

Glycosylation site: An amino acid sequence on the surface of a polypetide, such as a protein, which accommodates the attachment of a glycan, A N-linked glycosylation site is triplet sequence of NXS/T in which N is asparagine, X is any residues except proline, S/T means serine or threonine. A glycan is a polysaccharide or oligosaccharide. Glycan may also be used to refer to the carbohydrate portion of a glycoconjugate, such as a glycoprotein, glycolipid, or a proteoglycan.

gp120: An envelope protein from human immunodeficiency virus (HIV). The envelope protein is initially synthesized as a longer precursor protein of 845-870 amino acids in size, designated gp160. The gp160 forms a homotrimer and undergoes glycosylation within the Golgi apparatus. It is then cleaved by a cellular protease into gp120 and gp41. The gp41 contains a transmembrane domain and remains in a trimeric configuration; it interacts with gp120 in a non-covalent manner. The gp120 contains most of the external, surface-exposed, domains of the envelope glycoprotein complex, and it is gp120, which binds both to the cellular CD4 receptor and to the cellular chemokine receptors (such as CCR5).

The mature gp120 wild-type polypeptides have about 500 amino acids in the primary sequence. The gp120 is heavily N-glycosylated giving rise to an apparent molecular weight of 120 kD. The polypeptide is comprised of five conserved regions (C1-C5) and five regions of high variability (V1-V5). Exemplary sequence of wild-type gp160 polypeptides are shown on GENBANK®, for example Accession No. AAB05604 and AAD12142 which are incorporated herein by reference in their entirety as available on Feb. 7, 2008.

The gp120 core has a unique molecular structure, which comprises two domains: an “inner” domain (which faces gp41) and an “outer” domain (which is mostly exposed on the surface of the oligomeric envelope glycoprotein complex). The two gp120 domains are separated by a “bridging sheet” that is not part of either of these domains. The gp120 core comprises 25 beta strands, 5 alpha helices, and 10 defined loop segments.

The CD4-bound state of gp120 comprises an inner domain, an outer domain and a four-stranded bridging sheet mini-domain. The deglycosylated core of gp120 as dissected from the ternary complex approximates a prolate ellipsoid with dimensions of 50×50×25 A, although its overall profile is more heart-shaped than circular. This core gp120 comprises 25 (3-strands, 5 α-helices and 10 defined loop segments. The polypeptide chain of gp120 is folded into two major domains, plus certain excursions that emanate from this body. The inner domain (inner with respect to the N and C termini) features a two-helix, two-strand bundle with a small five-stranded β-sandwich at its termini-proximal end and a projection at the distal end from which the V1/V2 stem emanates. The outer domain is a stacked double barrel that lies alongside the inner domain so that the outer barrel and inner bundle axes are approximately parallel.

The bridging sheet (β3, β2, β21, β20) packs primarily over the inner domain, although some surface residues of the outer domain, such as Phenylalanine 382, reach in to form part of its hydrophobic core.

The gp120 polypeptides also include “gp120-derived molecules” which encompasses analogs (non-protein organic molecules), derivatives (chemically functionalized protein molecules obtained starting with the disclosed protein sequences) or mimetics (three-dimensionally similar chemicals) of the native gp120 structure, as well as proteins sequence variants (such as mutants), genetic alleles, fusions proteins of gp120, antigens, or combinations thereof.

The numbering used in the gp120 derived antigens disclosed herein is relative to the HXB2 numbering scheme as set forth in Numbering Positions in HIV Relative to HXB2CG Bette Korber et al., Human Retroviruses and AIDS 1998: A Compilation and Analysis of Nucleic Acid and Amino Acid Sequences. Korber et al., Eds. Theoretical Biology and Biophysics Group, Los Alamos National Laboratory, Los Alamos, N. Mex., which is incorporated by reference herein in its entirety.

Homologous proteins: Proteins from two or more species that have a similar structure and function in the two or more species. For example a gp120 antigen from one species of lentivirus such as HIV-1 is a homologous antigen to a gp120 antigen from a related species such as HIV-2 or SIV. Homologous proteins share the same protein fold and can be considered structural homologs. Homologous proteins share a high degree of sequence conservation, such as at least 30% at least 40% at least 50%, at least 605, at least 70%, at least 80% or at least 90% sequence conservation. Homologous proteins can share a high degree of sequence identity, such as at least 30% at least 40% at least 50%, at least 60%, at least 70%, at least 80% or at least 90% sequence identity.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immunogenic polypeptide: A protein or a portion thereof that is capable of inducing an immune response in a mammal, such as a mammal infected or at risk of infection with a pathogen. Administration of an immunogenic polypeptide derived from a pathogen of interest that inducing an immune response. Administration of an immunogenic polypeptide can lead to protective immunity against a pathogen of interest. In some examples, an immunogenic polypeptide is an antigen that is antigenically-cloaked to focus immunogenicity to a target epitope. An “immunogenic gp120 polypeptide” is a gp120 molecule, an antigenically cloaked gp120 molecule, or a portion thereof that is capable of inducing an immune response in a mammal, such as a mammal with or without an HIV infection. Administration of an immunogenic gp120 polypeptide that induces an immune response can lead to protective immunity against HIV. In some examples, an immunogenic gp120 polypeptide is a disclosed antigen that is antigenically-cloaked to focus immunogenicity to a target epitope.

Immunogenic surface: A surface of a molecule, for example a protein such as a gp120 protein or polypeptide, capable of eliciting an immune response. An immunogenic surface includes the defining features of that surface, for example the three-dimensional shape and the surface charge. In some examples, an immunogenic surface is defined by the amino acids on the surface of a protein or peptide that are in contact with an antibody, such as a neutralizing antibody, when the protein and the antibody are bound together. A target epitope includes an antigenic surface.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogenic composition: A composition comprising an immunogenic peptide that induces a measurable CTL response against virus expressing the immunogenic peptide, or induces a measurable B cell response (such as production of antibodies) against the immunogenic peptide. In one example, an “immunogenic composition” is composition includes a disclosed antigen derived form a gp120 peptide that induces a measurable CTL response against virus expressing gp120 polypeptide, or induces a measurable B cell response (such as production of antibodies) against a gp120 polypeptide. It further refers to isolated nucleic acids encoding an antigen, such as a nucleic acid that can be used to express the antigen (and thus be used to elicit an immune response against this polypeptide).

For in vitro use, an immunogenic composition may consist of the isolated protein, peptide epitope, or nucleic acid encoding the protein, or peptide epitope. For in vivo use, the immunogenic composition will typically comprise the protein or immunogenic peptide in pharmaceutically acceptable carriers, and/or other agents. Any particular peptide, such as disclosed antigen or a nucleic acid encoding the antigen, can be readily tested for its ability to induce a CTL or B cell response by art-recognized assays. Immunogenic compositions can include adjuvants, which are well known to one of skill in the art.

Immunologically reactive conditions: Includes reference to conditions which allow an antibody raised against a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. The immunologically reactive conditions employed in the methods are “physiological conditions” which include reference to conditions (such as temperature, osmolarity, pH) that are typical inside a living mammal or a mammalian cell. While it is recognized that some organs are subject to extreme conditions, the intra-organismal and intracellular environment is normally about pH 7 (such as from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

Inhibiting or treating a disease: Inhibiting the full development of a disease or condition, for example, in a subject who is at risk for a disease such as an infection with a pathogen, for example a bacterial, fungal or viral pathogen, such as HIV. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a protein, for example a disclosed antigen or nucleic acid encoding such an antigen) has been substantially separated or purified away from other biological components in which the component naturally occurs, such as other chromosomal and extrachromosomal DNA, RNA, and proteins. Proteins, peptides and nucleic acids that have been “isolated” include proteins purified by standard purification methods. The term also embraces proteins or peptides prepared by recombinant expression in a host cell as well as chemically synthesized proteins, peptides and nucleic acid molecules. Isolated does not require absolute purity, and can include protein, peptide, or nucleic acid molecules that are at least 50% isolated, such as at least 75%, 80%, 90%, 95%, 98%, 99%, or even 100% isolated.

K_{d}: The dissociation constant for a given interaction, such as a polypeptide ligand interaction or an antibody antigen interaction. For example, for the bimolecular interaction of an antibody (such as b12) and an antigen (such as gp120) it is the concentration of the individual components of the bimolecular interaction divided by the concentration of the complex.

Label: A detectable compound or composition that is conjugated directly or indirectly to another molecule to facilitate detection of that molecule. Specific, non-limiting examples of labels include fluorescent tags, enzymatic linkages, and radioactive isotopes. In some examples, a disclosed antigen is labeled with a detectable label.

Ligand: Any molecule which specifically binds a protein, such as a gp120 protein, and includes, inter alia, antibodies that specifically bind a gp120 protein. In alternative embodiments, the ligand is a protein or a small molecule (one with a molecular weight less than 6 kiloDaltons).

Naturally Occurring Amino Acids: L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, gamma.-carboxyglutamic acid, arginine, ornithine and lysine. Unless specifically indicated, all amino acids referred to in this application are in the L-form. “Synthetic amino acids” refers to amino acids that are not naturally found in proteins. Examples of synthetic amino acids used herein, include racemic mixtures of selenocysteine and selenomethionine. In addition, unnatural amino acids include the D or L forms of nor-leucine, para-nitrophenylalanine, homophenylalanine, para-fluorophenylalanine, 3-amino-2-benzylpropionic acid, homoarginine, and D-phenylalanine. The term “positively charged amino acid” refers to any naturally occurring or synthetic amino acid having a positively charged side chain under normal physiological conditions. Examples of positively charged naturally occurring amino acids are arginine, lysine and histidine. The term “negatively charged amino acid” refers to any naturally occurring or synthetic amino acid having a negatively charged side chain under normal physiological conditions. Examples of negatively charged naturally occurring amino acids are aspartic acid and glutamic acid. The term “hydrophobic amino acid” refers to any amino acid having an uncharged, nonpolar side chain that is relatively insoluble in water. Examples of naturally occurring hydrophobic amino acids are alanine, leucine, isoleucine, valine, proline, phenylalanine, tryptophan and methionine. The term “hydrophilic amino acid” refers to any amino acid having an uncharged, polar side chain that is relatively soluble in water. Examples of naturally occurring hydrophilic amino acids are serine, threonine, tyrosine, asparagine, glutamine, and cysteine.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide. A gp120 polynucleotide is a nucleic acid encoding a gp120 polypeptide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Peptide Modifications: Peptides can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains can be converted to C₁-C₁₆ alkoxy or to a C₁-C₁₆ ester using well-recognized techniques. Phenyl and phenolic rings of the peptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability. For example, a C- or N-terminal cysteine can be added to the peptide, so that when oxidized the peptide will contain a disulfide bond, generating a cyclic peptide. Other peptide cyclizing methods include the formation of thioethers and carboxyl- and amino-terminal amides and esters.

Peptidomimetic and organomimetic embodiments are also within the scope of the present disclosure, whereby the three-dimensional arrangement of the chemical constituents of such peptido- and organomimetics mimic the three-dimensional arrangement of the peptide backbone and component amino acid side chains, resulting in such peptido- and organomimetics of the proteins of this disclosure. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included within the scope of the disclosure are mimetics prepared using such techniques. In one example, a mimetic mimics the antigenic activity generated by gp120 a mutant, a variant, fragment, or fusion thereof.

Peptide: Any compound composed of amino acids, amino acid analogs, chemically bound together. Peptide as used herein includes oligomers of amino acids, amino acid analog, or small and large peptides, including polypeptides or proteins. Any chain of amino acids, regardless of length or post-translational modification (such as glycosylation or phosphorylation). “Peptide” applies to amino acid polymers to naturally occurring amino acid polymers and non-naturally occurring amino acid polymer as well as in which one or more amino acid residue is a non-natural amino acid, for example a artificial chemical mimetic of a corresponding naturally occurring amino acid. A “residue” refers to an amino acid or amino acid mimetic incorporated in a polypeptide by an amide bond or amide bond mimetic. A peptide has an amino terminal (N-terminal) end and a carboxy terminal (C-terminal) end.

A “protein” is a peptide that folds into a specific three-dimensional structure. A protein can include surface exposed amino acid resides and non-surface exposed amino acid resides. “Surface exposed amino acid residues” are those amino acids that have some degree of exposure on the surface of the protein, for example such that they can contact the solvent when the protein is in solution. In contrast, non-surface exposed amino acids are those amino acid residues that are not exposed on the surface of the protein, such that they do not contact solution when the protein is in solution. In some examples, the non-surface exposed amino acid residues are part of the protein core.

A “protein core” is the interior of a folded protein, which is substantially free of solvent exposure, such as solvent in the form of water molecules in solution. Typically, the protein core is predominately composed of hydrophobic or apolar amino acids. In some examples, a protein core may contain charged amino acids, for example aspartic acid, glutamic acid, arginine, and/or lysine. The inclusion of uncompensated charged amino acids (a compensated charged amino can be in the form of a salt bridge) in the protein core can lead to a destabilized protein. That is, a protein with a lower Tₘ then a similar protein without an uncompensated charged amino acid in the protein core. In other examples, a protein core may have a cavity within the protein core. Cavities are essentially voids within a folded protein where amino acids or amino acid side chains are not present. Such cavities can also destabilize a protein relative to a similar protein without a cavity. Thus, when creating a stabilized form of a protein, it may be advantageous to substitute amino acid residues within the core in order to fill cavities present in the wild-type protein.

Amino acids in a peptide, polypeptide or protein generally are chemically bound together via amide linkages (CONH). Additionally, amino acids may be bound together by other chemical bonds. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci pp. 463-468, 1980; Hudson, et al., Int J Pept Prot Res 14:177-185, 1979; Spatola et al. Life Sci 38:1243-1249, 1986; Harm J. Chem. Soc Perkin Trans. 1307-314, 1982; Almquist et al. J. Med. Chem. 23:1392-1398, 1980; Jennings-White et al. Tetrahedron Lett 23:2533, 1982; Holladay et al. Tetrahedron. Lett 24:4401-4404, 1983; and Hruby Life Sci 31:189-199, 1982.

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of the proteins and other compositions herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions, powder, pill, tablet, or capsule forms, conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polyclonal sera: Sera obtained from a subject immunized with an antigen of interest that contains more than one antibody, for example, an antibody that specifically binds a target epitope of a protein and other antibodies that specifically bind epitopes other than the target epitope.

Purified: The term purified does not require absolute purity; rather, it is intended as a relative term. Thus, for example, a purified protein is one in which the protein is more enriched than the protein is in its natural environment within a cell. Preferably, a preparation is purified such that the protein represents at least 50% of the protein content of the preparation.

The immunogenically-cloaked immunogens disclosed herein, or antibodies that specifically bind the disclosed immunogenically-cloaked immunogens, can be purified by any of the means known in the art. See for example Guide to Protein Purification, ed. Deutscher, Meth. Enzymol. 185, Academic Press, San Diego, 1990; and Scopes, Protein Purification: Principles and Practice, Springer Verlag, New York, 1982. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

For sequence comparison of nucleic acid sequences and amino acids sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)). The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993 and Ausubel et al. Short Protocols in Molecular Biology, 4^{th} ed., John Wiley & Sons, Inc., 1999.

“Stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid.

Specifically bind: When referring to an antibody, refers to a binding reaction which determines the presence of a target protein, peptide, or polysaccharide in the presence of a heterogeneous population of proteins and other biologics. Thus, under designated conditions, an antibody binds preferentially to a particular target protein, peptide or polysaccharide (such as an antigen present on the surface of a pathogen, for example gp120) and do not bind in a significant amount to other proteins or polysaccharides present in the sample or subject. Specific binding can be determined by methods known in the art. With reference to an antibody antigen complex, specific binding of the antigen and antibody has a K_{d} of less than about 10⁻⁶ Molar, such as less than about 10⁻⁶ Molar, 10⁻⁷ Molar, 10⁻⁸ Molar, 10⁻⁹, or even less than about 10⁻¹⁰ Molar.

Substitution: The replacement of one thing with another. With reference to an amino acid in a polypeptide “substitution” means replacement of one amino acid with a different amino acid. With reference to a nucleotide in a nucleic acid sequence “substitution” means replacement of one nucleotide with a different nucleotide.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cells is a cytotoxic T lymphocytes. In another embodiment, a CD8 cell is a suppressor T cell.

Therapeutic agent: Used in a generic sense, it includes treating agents, prophylactic agents, and replacement agents.

Therapeutically effective amount: A quantity of a specific substance (for example a disclosed antigen) sufficient to achieve a desired effect in a subject being treated. For instance, this can be the amount necessary to inhibit or treat an infection by a pathogen, such as an infection by a bacterial pathogen. When administered to a subject, a dosage will generally be used that will achieve target tissue concentrations shown to achieve a desired in vitro effect.

A therapeutically effective amount of a substance, such as an antigen, can be administered in a single dose, or in several doses, for example daily, during a course of treatment. However, the effective amount of a composition will be dependent on the compound applied, the subject being treated, the severity and type of the affliction, and the manner of administration of the composition. For example, a therapeutically effective amount of composition can vary from about 0.01 mg/kg body weight to about 1g/kg body weight.

Tₘ: The temperature at which a change of state occurs. For example, the temperature at which protein such as a gp120 undergoes a transition from the folded form to the unfolded form. Essentially this is the temperature at which the structure melts away. Another example would be the temperature at which a DNA duplex melts.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of DNA by electroporation, lipofection, and particle gun acceleration.

Transmembrane domain: An amino acid sequence that inserts into a lipid bilayer, such as the lipid bilayer of a cell or virus like particle. A transmembrane domain can be used to anchor a antigen to a membrane. In some examples a transmembrane domain is a gp41 transmembrane domain. In some examples the transmembrane domain in a hCD4 transmembrane domain.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example, a bacterial or viral pathogen, or to a cellular constituent correlated with a pathological condition. A vaccine may include a polynucleotide (such as a nucleic acid encoding an a disclosed antigen), a peptide or polypeptide (such as a disclosed antigen), a virus, a bacteria, a cell or one or more cellular constituents.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

Virus: A virus consists essentially of a core of nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. For example, a viral infection may result in a cell producing a cytokine, or responding to a cytokine, when the uninfected cell does not normally do so. In some examples, a virus is a pathogen. Specific examples of viral pathogens for which an immune response can be generate in accordance with the disclosed methods include, without limitation; Arenaviruses (such as Guanarito virus, Lassa virus, Junin virus, Machupo virus and Sabia), Arteriviruses, Roniviruses, Astroviruses, Bunyaviruses (such as Crimean-Congo hemorrhagic fever virus and Hantavirus), Barnaviruses, Birnaviruses, Bornaviruses (such as Boma disease virus), Bromoviruses, Caliciviruses, Chrysoviruses, Coronaviruses (such as Coronavirus and SARS), Cystoviruses, Closteroviruses, Comoviruses, Dicistroviruses, Flaviruses (such as Yellow fever virus, West Nile virus, Hepatitis C virus, and Dengue fever virus), Filoviruses (such as Ebola virus and Marburg virus), Flexiviruses, Hepeviruses (such as Hepatitis E virus), human adenoviruses (such as human adenovirus A-F), human astroviruses, human BK polyomaviruses, human bocaviruses, human coronavirus (such as a human coronavirus HKU1, NL63, and OC43), human enteroviruses (such as human enterovirus A-D), human erythrovirus V9, human foamy viruses, human herpesviruses (such as human herpesvirus 1 (herpes simplex virus type 1), human herpesvirus 2 (herpes simplex virus type 2), human herpesvirus 3 (Varicella zoster virus), human herpesvirus 4 type 1 (Epstein-Barr virus type 1), human herpesvirus 4 type 2 (Epstein-Barr virus type 2), human herpesvirus 5 strain AD169, human herpesvirus 5 strain Merlin Strain, human herpesvirus 6A, human herpesvirus 6B, human herpesvirus 7, human herpesvirus 8 type M, human herpesvirus 8 type P and Human Cyotmegalovirus), human immunodeficiency viruses (HIV) (such as HIV 1 and HIV 2), human metapneumoviruses, human papillomaviruses (such as human papillomavirus-1, human papillomavirus-18, human papillomavirus-2, human papillomavirus-54, human papillomavirus-61, human papillomavirus-cand90, human papillomavirus RTRX 7, human papillomavirus type 10, human papillomavirus type 101, human papillomavirus type 103, human papillomavirus type 107, human papillomavirus type 16, human papillomavirus type 24, human papillomavirus type 26, human papillomavirus type 32, human papillomavirus type 34, human papillomavirus type 4, human papillomavirus type 41, human papillomavirus type 48, human papillomavirus type 49, human papillomavirus type 5, human papillomavirus type 50, human papillomavirus type 53, human papillomavirus type 60, human papillomavirus type 63, human papillomavirus type 6b, human papillomavirus type 7, human papillomavirus type 71, human papillomavirus type 9, human papillomavirus type 92, and human papillomavirus type 96), human parainfluenza viruses (such as human parainfluenza virus 1-3), human parechoviruses, human parvoviruses (such as human parvovirus 4 and human parvovirus B 19), human respiratory syncytial viruses, human rhinoviruses (such as human rhinovirus A and human rhinovirus B), human spumaretroviruses, human T-lymphotropic viruses (such as human T-lymphotropic virus 1 and human T-lymphotropic virus 2), Human polyoma viruses, Hypoviruses, Leviviruses, Luteoviruses, Lymphocytic choriomeningitis viruses (LCM), Marnaviruses, Narnaviruses, Nidovirales, Nodaviruses, Orthomyxoviruses (such as Influenza viruses), Partitiviruses, Paramyxoviruses (such as Measles virus and Mumps virus), Picornaviruses (such as Poliovirus, the common cold virus, and Hepatitis A virus), Potyviruses, Poxviruses (such as Variola and Cowpox), Sequiviruses, Reoviruses (such as Rotavirus), Rhabdoviruses (such as Rabies virus), Rhabdoviruses (such as Vesicular stomatitis virus, Tetraviruses, Togaviruses (such as Rubella virus and Ross River virus), Tombusviruses, Totiviruses, Tymoviruses, and Noroviruses among others.

“Retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2), simian immunodeficiency virus (SIV), and feline immunodeficiency virus (FIV).

HIV-1 is a retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by an HIV virus, as determined by antibody or western blot studies. Laboratory findings associated with this disease are a progressive decline in T cells.

Wild-type antigen: An antigen that has not been modified by selective mutation to focus that antigenicity of the antigen to a target epitope.

### II. Description of Several Embodiments

Because many epitopes are derived from different and non-contiguous regions of the primary amino acid sequence of the antigen, it has proven difficult to synthetically reproduce these complex epitopes.

To overcome this problem, disclosed herein are methods for antigenic cloaking of an antigen by selecting a target epitope on the surface of the antigen and altering surface residues outside of the selected target epitope. The altered surface residue reduce the production of antibodies to the non-target epitopes of the antigen that do not produce neutralizing antibodies and/or produce antibodies that interfere with an antibody that binds to the target epitope. Thus, the methods of the current disclosure allow for the production of antigens in which the antigenicity is focused to the selected target epitope.

Antigenic cloaking involves the structure-based variation of the antigenic surfaces outside of a target epitope (the selected antigenic surface). An appropriately cloaked molecule retains binding capacity to the antibody of interest (such as an antibody that specifically binds the target epitope), but not to antibodies that do not recognize the target epitope. In other words, by selectively mutating the surface residues of an antigen that are not part of a target epitope, the antigenic properties of the antigen can be focused to a specific target epitope that includes a selected antigenic surface. Such a molecule would have utility as a diagnostic (for example to detect and quantify target antibodies in a polyclonal serum response) and also as an immunogen (for example to immune-focus the immune response to the epitope defined by the target epitope).

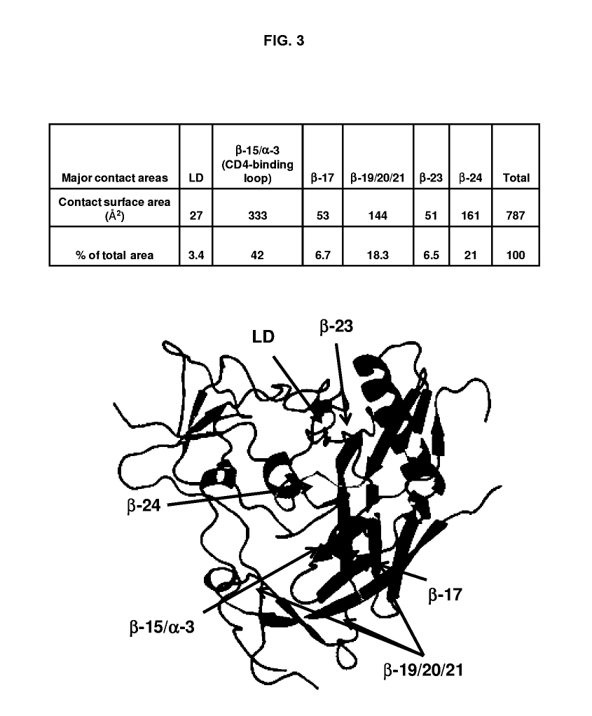

With specific reference to HIV, the disclosed methods now provides a means by which an effective HIV-1 vaccine might be obtained. Past efforts have been to analyze is by analyzing antibodies that can broadly and potently neutralize HIV-1, define their epitopes, and then re-elicit such antibodies with appropriate epitope mimetics. However, because many of the epitopes include discrete non-continuous regions of primary sequence, design of synthetic epitope mimetics has met with little success. For example, one of the most promising antibodies is the b12 antibody, which binds to an epitope comprised of residues from 6 different discrete portions of the gp120 sequence (see FIG. 3). As disclosed herein using methods of computation designed based on the X-ray crystallographic analysis of gp120, antigens have been designed that focus antigenicity to the b12 epitope.

While the methods of this disclosure have been applied herein to the HIV-1 gp120 protein, the methods are equally applicable to an antigen from any pathogen of interest, such as a viral, bacterial, or fungal pathogen, for which a broadly neutralizing antibody and its respective epitope have been characterized at the atomic-level. Such antigens may serve as the basis of effective therapies, such as HIV-1 vaccines. They may also serve as valuable diagnostics, for example, to specifically identify serum reactivities against target HIV-1 epitopes.

A. Methods for Computational Design of Antigenically-cloaked Antigens

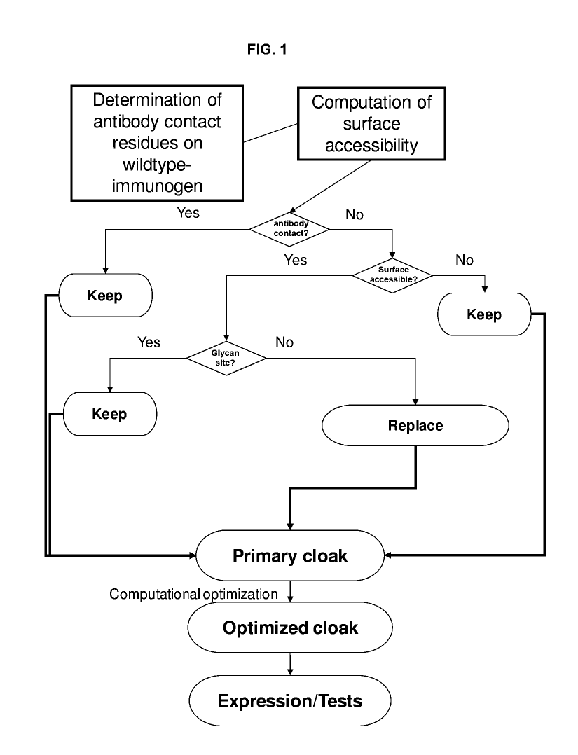

A computational method for antigenic cloaking is disclosed herein. A design flow chart for the general method of producing an antigen that has been antigenically-cloaked is shown in FIG. 1. It is understood that the steps may be performed in any order and one or more of the steps may be repeated or omitted as necessary. With reference to FIG. 1, the atomic coordinates of an antigen of interest are obtained for which the amino acid residues that contact an antibody of interest (such as neutralizing antibody) are known. In some examples, the amino acid residues of the antigen that contact the antibody (contact residues) can be determined from the atomic coordinates of a complex between the antigen and an antibody. The contact residues can also be obtained more indirectly by epitope-mapping studies such as alanine-scanning or hydrogen-deuterium exchange, so the structure of the antibody/antigen complex is not required although it is advantageous.

In some examples, the atomic coordinates are structural data obtained by X-ray crystallographic or nuclear magnetic resonance methods. In some examples, the atomic coordinates are contained within a computer searchable database. In one example, such a database includes at least a portion of the Protein Data Bank (PDB) (available on the world wide web at rcsb.org). The PDB is a publicly available depository of information about the three-dimensional structures of large biological molecules, including but not limited to, proteins and nucleic acids. A variety of information associated with each structure is available through the PDB, including sequence details, atomic coordinates, crystallization conditions; 3-D structure neighbors computed using various methods, derived geometric data, structure factors, 3-D images and a variety of links to other resources. While the PDB represents the greatest number of compiled atomic coordinates of biological molecules, atomic coordinates may be obtained from any other appropriate resource, such as private databases and research data.

With continued reference to FIG. 1, because the methods disclosed herein use the atomic coordinates of the antigen of interest, surface exposed amino acid residues of the antigen can be determined, for example, by determining the surface accessibility of the amino acid in the antigen, for example using computer programs such as GRASP Nicholls et al., Proteins, Struct. Funct. Gen. 1991, 11, 282, MS (Connolly Science, 221, 709-713, 1983) or NACCESS (Hubbard, S. J.& Thornton, J. M. (1993), ‘NACCESS’, Computer Program, Department of Biochemistry and Molecular Biology, University College London). In some examples, a surface exposed amino acid residue is defined as a as residues with greater than 30% sidechain surface area exposed, relative to the same sidechain in an isolated tripeptide, for example, greater than 30%, greater than 35%, greater than 40%, greater than 45%, greater than 50%, greater than 55%, greater than 60%, greater than 65%, greater than 70%, greater than 75%, greater than 80%, greater than 85% greater than 90%, or even greater than 95% sidechain surface area exposed, relative to the same sidechain in an isolated tripeptide. The amino acids that are not surface accessible can be maintained, although in certain situations it may be advantageous to selectively mutate a few non-surface accessible amino acids (such as less than 10%, less than 9%, less than 8%, less than 7%, less than 6%, less than 5%, less than 4%, less than 3%, less than 2%, or less than 1%), for example, to increase antigen stability (see, for example, International Patent Publication WO 07/030,518 incorporated by reference herein in its entirety). Proteins generally fold into unique three-dimensional structures which are primarily dependent on the amino acid residues in the core of protein (for example the non-surface accessible amino acids residues). Thus, keeping the non-surface exposed amino acid residues even when surface exposed amino acid residues are mutated is believed to result in an antigen that maintains the same fold and overall three-dimensional structure as the wild-type antigen.

The surface exposed amino acid residues that contact the antibody define a target epitope that includes an antigenic surface. The surface exposed amino acid residues that contact the antibody can be determined from examination of the atomic coordinates of a an antigen antibody complex, the mapping of alanine scanning mutagenesis, hydrogen deuterium exchange or other technique. The surface exposed amino acid residues of the antigen that contact the antibody are maintained and are not selected for mutation. In some examples, the amino acid residues that contact the antibody are defined any amino acid residue in the antigen as having at least one heavy-atom (non-hydrogen atom) within about 10.0 angstroms (such as within about 9.5, about 9.0, about 8.0, about 7.5, about 7.0, about 6.5, about 6.0, about 5.5, about 5.0, about 4.5, about 4.0, or about 3.5 angstroms) of a heavy-atom on the antibody. In later generations of cloaked molecules, the definition of contact residues could be narrowed. Conversely, the surface exposed amino acid residues of the antigen that do not contact the antibody are sites of possible mutation (for example that do not have a heavy atom within about 10.0 angstroms) of a heavy-atom on the antibody (such as within about 9.5, about 9.0, about 8.0, about 7.5, about 7.0, about 6.5, about 6.0, about 5.5, about 5.0, about 4.5, about 4.0, or about 3.5 angstroms). These surface exposed amino acid residues of the antigen that do not contact the antibody are further queried to determine if they represent a glycosylation site. If the surface exposed amino acid residues of the antigen that do not contact the antibody represent a glycosylation site they are maintained. The remaining surface exposed amino acid residues of the antigen that do not contact the antibody are selected as possible sites of mutation. In some examples between about 10% and about 100, such as about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or even 100%, such as 10% to about 20%, 20% to about 40%, 10% to about 50%, 30% to about 50%, 40% to about 70%, 60% to about 90%, about 50 to about 100% , or 20% to about 70% of the surface exposed amino acid residues of the antigen that do not contact the antibody are selected for mutation These surface exposed amino acid residues of the antigen that do not contact the antibody are the positions at which a computational design simulation can select optimal combinations of amino acids to “cloak” the antigen while maintaining folding stability and solubility (methods of determining protein stability and solubility are well known in the art). The antigen may be further modified following computational optimization. Ultimately, the antigen is evaluated by expression and functional testing.

In certain embodiments, protein and design calculations are performed using the ROSETTADESIGN computer program, available from the University of Washington ROSETTADESIGN (Kuhlman et al. (2003). Science, 302, 1364-1368; Kuhlman et al Proc Natl Acad Sci USA. 2000 Sep. 12; 97(19):10383-8). ROSETTADESIGN is a software application which provides protein structure predictions. ROSETTADESIGN utilizes physical models of the macromolecular interactions and algorithms for finding the lowest energy structure for an amino acid sequence in order to predict the structure of a protein. Furthermore, ROSETTADESIGN may use these models and algorithms to find the lowest energy amino acid sequence for a protein or protein-protein complex for protein design. Multiple energetically acceptable “cloaked” antigens can be produced in such simulations, owing to the freedom to accommodate a wide variety of side-chains and side-chain conformations on the solvent-accessible surfaces of proteins while maintaining folding stability and solubility.

In some examples, the side chains of the epitope-scaffold and the antibody are allowed to vary between discrete rotamers. The rotamers are selected from a backbone dependent rotamers library. Non-limiting examples of such a library have been disclosed by R. L. Dunbrack and F. E. Cohen (Protein Sci 6, p. 1661-, August 1997) and B. Kuhlman and D. Baker (Proc Natl Acad Sci USA 97, 10383, Sep. 12, 2000). These libraries provide information on the possible side chains for amino acids, including the statistical preferences in bond angles for the proteins and how changes in one angle tend to affect other angles. Thus, calculations can be restricted to high probability, relatively stable rotamers, rather than those rotamers which are much less probable and less stable. As a result, the calculations are made easier, as there are less calculations to perform, and the results are likely to be more stable, since only relatively high probability rotamers are examined.

During the design process, available information from homologs of the antigen of interest can be incorporated during computational antigenic cloaking, for example, to assist in maintaining proper folding and solubility of cloaked constructs. In this scenario, a multiple sequence alignment could be constructed for the antigen of interest, and at each surface exposed non-contact surface position, the computational design simulation could be restricted to choose among the amino acids present in the sequence alignment for that position. There are many possible options for biasing the selection of amino acids in this case. For example, the design simulation could be biased at any position to favor amino acids that occur more frequently in the multiple sequence alignment for that position.

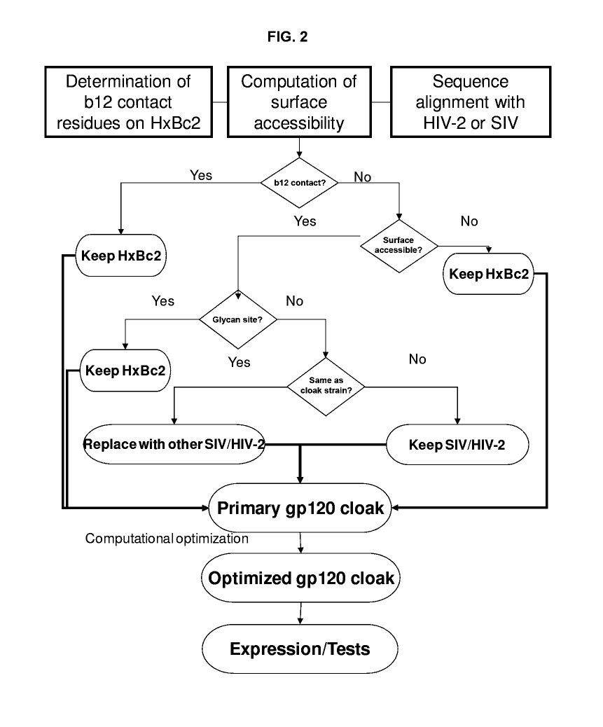

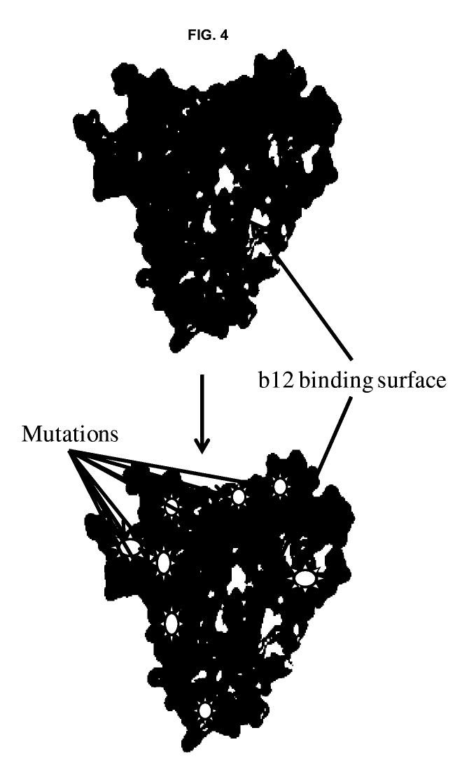

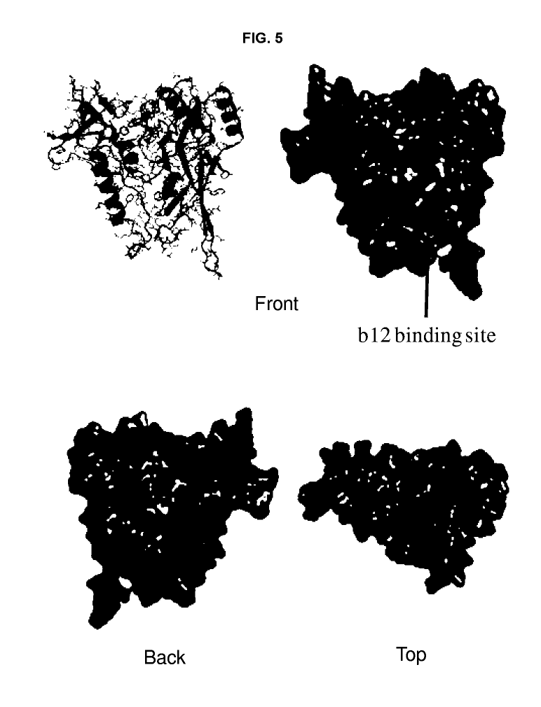

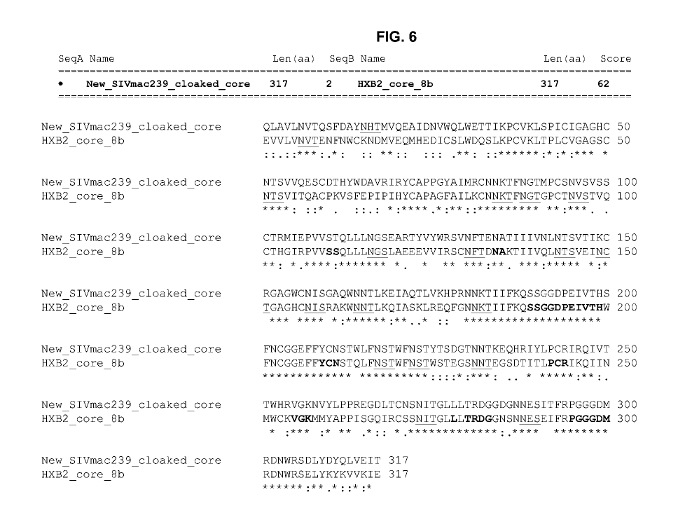

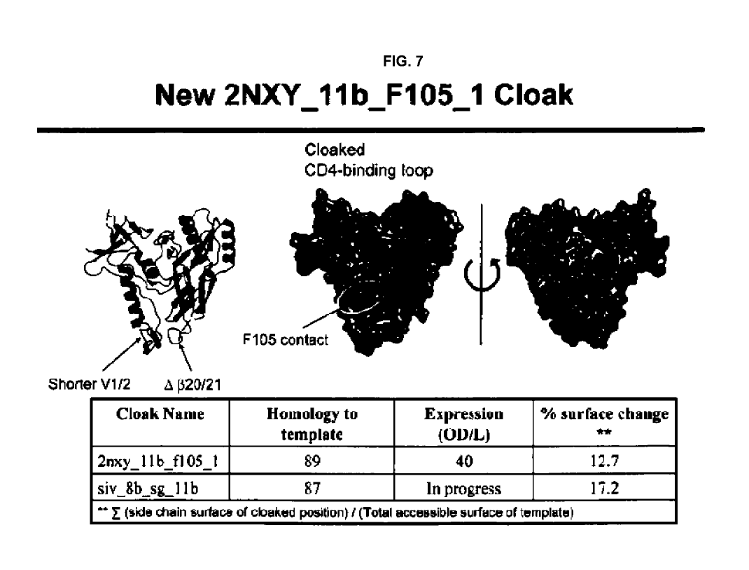

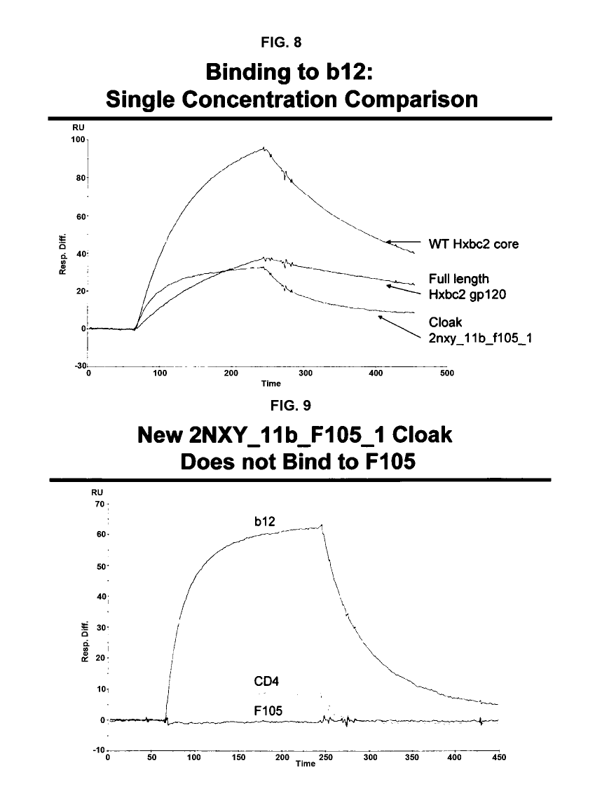

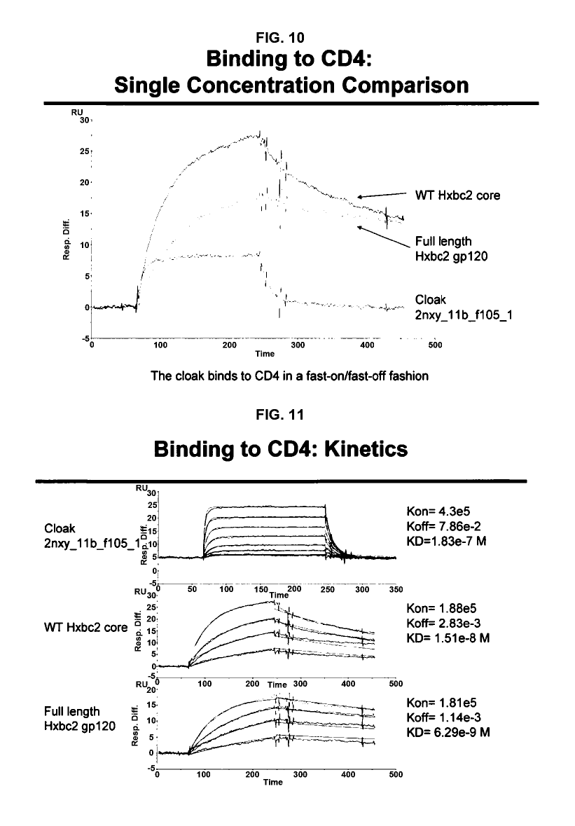

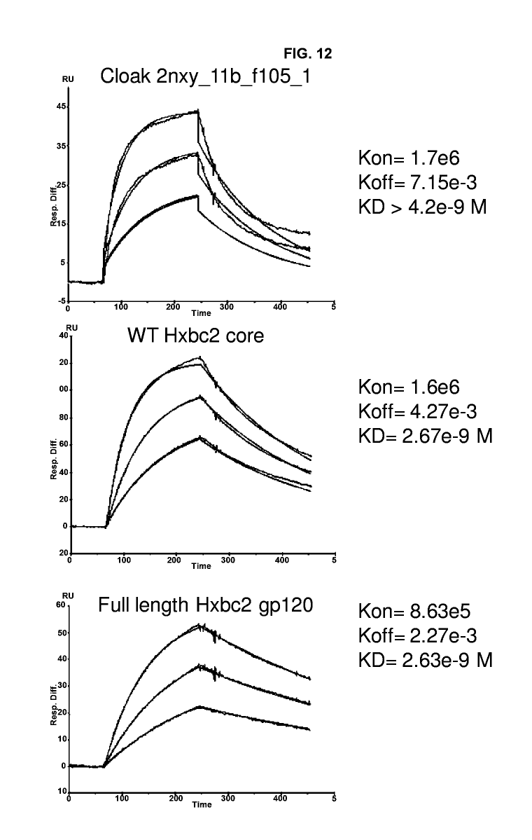

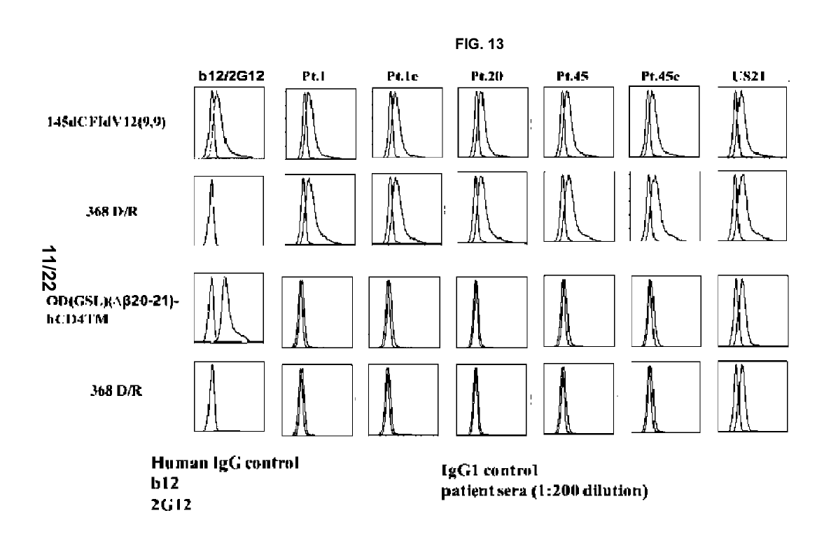

With specific reference to an antigen from HIV-1 (see FIG. 2), a sequence alignment of the antigen from HIV-1 and a homologous protein from another strain (for example, HIV-2 or SIV, termed cloaking strain) reveals differences in amino acid sequence. Initially, the residues on the HIV-1 epitope that are contacted by antibody (for example by b12) are identified (see for example FIG. 6) to define the target epitope. The surface and/or solvent accessibility of the contacted residues is calculated, for example using the program GRASP, MS or NACCESS. If a given amino acid residue in the HIV-1 epitope is contacted by the antibody, the amino acid residue is maintained. If it is not contacted, the surface exposure of the residue is taken into account. If the residue is not a surface exposed amino acid residue, it is maintained. If the residue is a surface exposed amino acid residue, potential glycosylation at the site is investigated. If the residue is part of a glycosylation site, it is maintained. If it is not part of a glycosylation site, the sequence alignment with the cloaking strain is consulted to determine if the residue is the same as in the cloaking strain. If the HIV-1 residue is the same as the cloaking strain, the residue is replaced with an amino acid from the cloaking strain (such as SIV or HIV-2 strain) that has a different amino acid at the position. If the HIV-1 residue is not the same as the cloaking strain, it is substituted with the corresponding residue in the cloaking strain (see, for example, FIGS. 4, 5 and 7).

To perform computational antigenic cloaking on glyco-proteins such as gp120, the native glycosylation sites could be maintained, or optionally the computational design can include introduction of glycosylation sites on the non-epitope surface. Design of an N-linked glycosylation site requires at minimum placing a triplet sequence of NXS/T on the protein in a location at which the N is solvent accessible, in which N is asparagine, X is any residues except proline, S/T means serine or threonine. With computational design of glycosylation sites, one can add and/or move glycans around on the non-target epitope surface, to enhance cloaking while maintaining folding and stability. Computational design of glycosylation sites is not limited to proteins that are already glycosylated one or more glycosylation sites can be designed on surface exposed non-contact amino acid residues as another application of antigenic cloaking. In some examples, amino acid residues of the antigen that contact NAG groups are maintained. In some examples, an amino acid residues of the antigen that contacting NAG group are defined as any residue with at least one sidechain heavy atom within about 8.0 angstroms of any heavy atom on the NAG group, such as within about 8.0, about 7.5, about 6.5, about 6.0, about 5.5, about 5.0, about 4.5, about 4.0, or about 3.5, angstroms of any heavy atom on the NAG group.

The antigens produced by the above methods may be subsequently examined in a post-design analysis. The post-design analysis may include gathering of additional information about the disclosed antigens and a manual analysis and redesign of the candidates. The rationale for the post-design analysis is that additional information can play an important role in selected which of the candidate disclosed antigens should be pursued in further testing. In one embodiment, one or more of the following types of information may be accumulated, as necessary: species origin, size, oligomerization state, number of disulfide bonds, average B-factor for backbone atoms, and hetero atoms present in the crystal structure. The oligomerization state, in certain embodiments, can be obtained from one of the RCSB Biological Unit Database and the Protein Quaternary Server at the European Bioinformatics Institutes Information.

This information can be used to prioritize disclosed antigens for further consideration, as well as to target selected immunogens for further processing. For example, if an immunogen is multimeric (dimeric, trimeric, etc.) then additional testing may be performed to determine if the oligomeric form of the antigen will clash with the antibody, for example inhibiting antibody binding. Alternatively, additional mutations may be performed to render the disclosed antigens monomeric.

In the post-design analysis, manual examination and redesign may also be performed. In one aspect, manual examination allows prioritization of disclosed antigens based on the accumulated post-design information. In another aspect, manual examination allows visual inspection and validation of disclosed antigens structural stability and epitope-antibody interaction. In a further aspect, manual examination may reveal that mutations back to wild-type may be implemented. Additional tests for stability and solubility can also be performed. The methods described herein can be performed by one pr more computers appropriately programmed. The methods described herein can also be stored on one or more computer readable media as computer executable instructions.

B. Antigenically-Cloaked Antigens

Antigens are disclosed herein that are antigenically-cloaked to target antigenicity of the antigen to a target epitope that specifically binds an antibody of interest, such as a neutralizing antibody. In some embodiments, the antigen is a bacterial, viral or fungal antigen, such as an antigen from one or more of the pathogens listed in the preceding Summary of Terms.

In some embodiments, the isolated antigens include a target epitope defined by atomic coordinates of those amino acids of the antigen that contact an antibody of interest that specifically binds the antigen. The disclosed antigens have been modified to substitute the surface exposed amino acids located exterior to the target epitope to focus the antigenicity of the antigen to the target epitope. For example, the method can remove non-target epitopes that might interfere with specific binding of an antibody to the target epitope. In some examples, the amino acid substitutions result in the antigen not being bound by antibodies in a polyclonal serum that specifically bind surface exposed amino acid residues of the wild-type antigen located exterior of the target epitope. In some embodiments, the amino acid substitutions alter antigenicity of the antigen in vivo as compared to the wild-type antigen (unsubstituted antigen) but do not introduce additional glycosylation sites as compared to the wild-type antigen. In some embodiments, that antigen is glycosylated.

The amino acid substitutions do not significantly alter the selective binding of the antigen to the antibody of interest. In other words, the antibody of interest will specifically bind the antigen with approximately the same affinity as the antibody of interest specifically binds that wild-type antibody, for example, as measured by dissociation constant (K_{d}) of the antibody of interest and the antigen and the antibody of interest and the wild-type antigen for example the substitutions that do not significantly alter the selective binding of an antibody to an antigen would be expected to alter the binding of the antigencally cloaked antigen less than 100 fold, such as less than 50 fold, less than 20 fold, less than 10 fold, less than 5 fold or even less than 2 fold. In some embodiments, the antibody of interest and the antigen have a K_{d} of less than about 1 μM, such as less than about, 100 nM, less than about 10 nM or even less than about 1 nM.

The disclosed antigens have amino acid substitutions between about 10% and about 100% of the surface exposed amino acid residues located exterior of the target epitope as compared to a wild-type antigen, such as about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or even 100%, such as 10% to about 20%, 20% to about 40%, 10% to about 50%, 30% to about 50%, 40% to about 70%, 60% to about 90%, about 50% to about 100%, 40% to about 100%, or 20% to about 70% of the surface exposed amino acid residues located exterior of the target epitope as compared to a wild-type antigen.

The disclosed antigens also have substitution of less than 10% of the non-surface exposed amino acid residues as compared to a wild-type antigen, such as less than about 10%, less than about 9%, less than about 8%, less than about 7%, less than about 6%, less than about 5%, less than about 4%, less than about 3%, less than about 2%, less than about 1% or even no amino acid substitutions of the non-surface exposed amino acid residues as compared to a wild-type antigen.

In some embodiments, the disclosed antigen is a human immunodeficiency virus (HIV)-1 antigen, such as a gp160, gp41, gp140 or gp120 antigen or an immunogenic fragment thereof and the antibody of interest is 2F5, 2G12, b12, or 4E10. In specific embodiments, the disclosed antigen is HIV-1 gp120 or an immunogenic fragment thereof, for example, the outer domain of gp120. In some examples, the outer domain of gp120 includes amino acid residues 252-482 of gp120.

In some example, the antigen is a multimer, such as a multimer of gp120, for example, a dimer, trimer, etc. of gp120 or an immunogenic fragment thereof. Several exogenous oligomerization motifs have been successfully used to promote stable trimers of soluble recombinant proteins: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. J Biol Chem 278:42200-42207), and the phage T4 fibritin foldon (Miroshnikov et al. 1998 Protein Eng 11:329-414). The fibritin foldon, a 27 amino acid sequence (GYIPEAPRDGQAYVRKDGEWVLLSTF, SEQ ID NO: 21), adopts a β-propeller conformation, and can fold and trimerize in an autonomous way (Tao et al. 1997 Structure 5:789-798). In some embodiments, the disclosed antigen includes one or more of a foldon domain (for example, to induce trimerization), a six-histadine residues tag (for example, to induce oligomerization and/or aid in purification) and a transmembrane domain (for example, to anchor the antigen in a cell membrane). In specific examples, the foldon domain is a T4 foldon domain. In some examples, the transmembrane domain is a gp41 transmembrane domain. In some examples the transmembrane domain is a hCD transmembrane domain. In specific examples a hCD transmembrane domain is set forth as the amino acid sequence LIVLGGVAGLLLFIGLGI (SEQ ID NO: 22).

In some examples, the mutations introduced on the surface exposed non-contact residues are mutations to the homologous residues in gp120 from SIV or HIV2 (see, for example, FIG. 6).

In some embodiments, a disclosed antigen includes the outer domain of a gp120 polypeptide that is appropriately mutated to provide antigenic cloaking. In some embodiments, the disclosed antigen is an antigenically-cloaked gp120 antigen in which the V1, V2, and/or V3 variable loops from gp120 are removed or truncated. In some examples v1/v2 and β β20/β21 regions to reduce the immunogenicity or at least alter the antigenicity of those regions. In some examples the β β20/β21 bridging sheet of the antigenically-cloaked gp120 antigen is removed, for example by replacing residues 422-436 of gp120 with Gly-Gly. In some examples the β β20/β21 bridging sheet of the antigenically-cloaked gp120 antigen is removed by replacing the amino acid residues between I423 and Y435 with Gly-Gly. n some examples, the residues 302-323 of gp120, part of the V3 loop, are replaced with a basic hexapeptide (NTRGRR, SEQ ID NO: 23). In some examples, the V3 loop is replaced with Gly-Val-Gly. The modified V1/V2 modification was taken from a core gp120 previously designed that had improved expression yields known as “new 9c” (see International Patent Publication NO. WO 2007/030518, which is incorporated herein by reference), and includes the insertion of VKLTPLAGATSVITQA (SEQ ID NO: 24) between C119 and C205. Though these modifications were not done using computational design, they do serve as examples of cloaking by modifying the backbone of the protein rather than just the sequence. Computational methods for flexible backbone protein design allow this more aggressive method of cloaking to be applied to loop-trimming as in the case of V1/V2 and β β20/β21, but also to trimming, modifying, or even building new backbone in more complex structural contexts.

Exemplary amino acid sequences of antigenically-cloaked gp120 antigens are given below as SEQ ID NOs: 1-9.

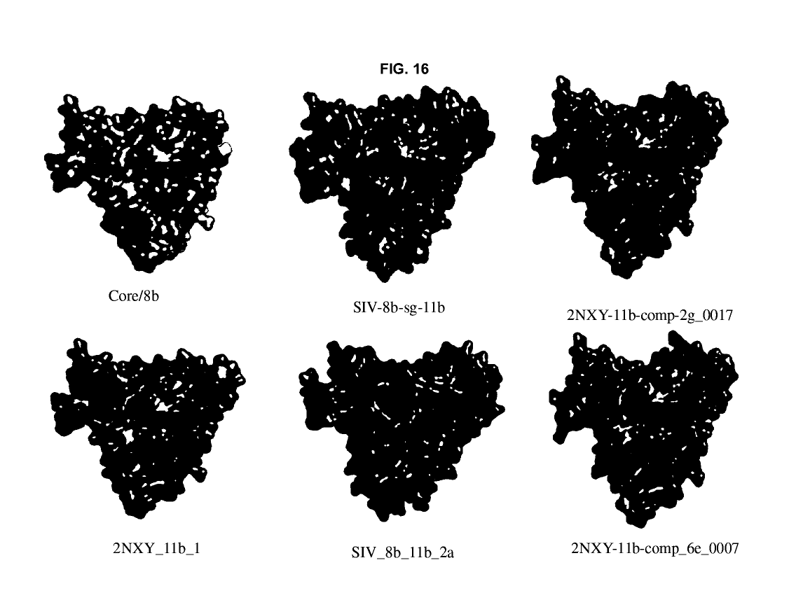

2NXY_{—}11b_{—}1:
Mpmgslqplatlyllgmlvasvlattylvnvtvtfdmwkndmveqmdeaiktlldtslkpcvkltplagatsvitqa cptvswepipirycappgyailkcnnktfngtgpctnvsvvtcthgirpvvssqllingsladeevvirsvnftdnakti ivqlntsveinctgaghcnitrakwnntlkqiaeklreqfgnnktinqssggdpeivthwfncggeffycnstqlfns twfnstwstkgsnntegsdtitlperikqiggyappvsgvitcssnitgllltrdggndnneseifrpgggdmrdnwrs elykykvvklegshhhhhh (SEQ ID NO: 1). A surface representation of the structure of the 2NXY_{—}11b_{—}1 antigen is shown in FIG. 16.
SIV-8b-sg-11b:
Mpmgslqplatlyllgmlvasvlattylvnvtvtfdwckndmvaqmntaictlwktsnkpcvkltplcvgagscnt svitqacptvsfepipirycappgyailkcnnktfngtgpctnvsvvtctdgirpvvssqllingtladeevvirscnftd naktiivqlntsveinctgaghcnitrakwnntlkqiaeklreqfgnnktinqssggdpeivthwfncggeffycnst qlfnstwfnstwstkgsnntegsdtitlperikqitgmwctvgkmmyappvsgvitcssnitgllltrdggndnnese ifrpgggdmrdnwrselykykvvkltgshhhhhh (SEQ ID NO: 2). A surface representation of the structure of the SIV-8b-sg-11b antigen is shown in FIG. 16.
SIV_{—}8b_{—}11b_{—}2a:
Mpmgslqplatlyllgmlvasvlattvtvnvtvtfdwcaddmvatmntaictlwktsndpctkcptvrfkpvpiryc appgyailkcnnrdfngtgpctnvsvvtctdgihpvvssqllingtladekvvirscnfsdnaktiivqlntsveinctg qghcnitrakwnqtlkqiaeklreqfgnnktiifrpssggdpeivthwfncggkffycnstqlfnstwfnstwstkgsn ntegsdtitlperirsitgmvctvgkmiyappvegvitcssnitgllltrdggndnneseifrpgggdmrdnwrselyk yrvvrltgshhhhhh (SEQ ID NO: 3). A surface representation of the structure of the SIV_{—}8b_{—}11b_{—}2a antigen is shown in FIG. 16.
SIV_{—}8b_{—}13_{—}2c:

Mpmgslqplatlyllgmlvasvlassysvnvtqtfswcdqdmvakmqqaicnlwqesdtpcndcptkafspqpi qycapngkailkcnnenfngtgpctnvsvvtctagispvvssqlllngeladetvvirscnfndnaktiivqlntsvein ctgeghcnitrakwnatlkqiakklrqqfgnnktiifqsssggdpeivthwfncggrffycnstqlfnstwfnstwstk gsnntegsdtislperiksitdmkcsvgkmiyappkagdikcssnitgllltrdggnnnneseifrpgggdmrdnwr selykyqvvelqgshhhhhh (SEQ ID NO: 4).

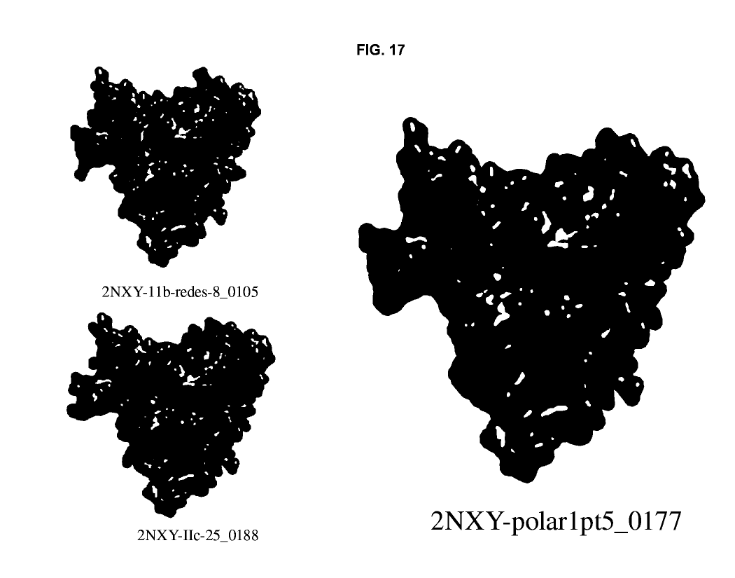

2NXY-11b-comp-2g_{—}0017:
Mpmgslqplatlyllgmlvasvlaqkylvnyteefnmwnnnmvelmhqkiaslikqslqpcvkltplagatsvit qacpkvdwepqpieycapdgfailkcnnstfngtgpctnvstvrcthgirpvvssqllingslassevvirsvnftdna ktiivqlntsveinctgdgrcniardkwnatlqqiasklrqqfgsnktiifeqssggdpeivthwfncggeffycnstqlf nstwfnstwstegsnntegsdtislperikqiggyapptrgqircssnitgllltrdggdssneseifrpgggdmrdnwr selykykvtpiegshhhhhh (SEQ ID NO: 5). A surface representation of the structure of the 2NXY-11b-comp-2g_{—}0017 antigen is shown in FIG. 16.
2NXY-11b-comp_{—}6e_{—}0007:
Mpmgslqplatlyllgmlvasvlarevlvnyteqfnmwmqmveamhreierleraklnpcvkltplagatsvitq acpkvqfeptpitycapegfailkcnndtfngtgpctnvstvdcthgirpvissqlllngslakgevvirsvnftdnakti ivqlntsveinctgrgycniarkkwnetlegiasklidqfgknktiifsqssggdpeivthwfncggeffycnstqlfns twfnstwstegsnntegsdtitlperikqiggyappqngqircssnitgllltrdggpsqneseifrpgggdmrdnwrs elykykvkaiegshhhhhh (SEQ ID NO: 6). A surface representation of the structure of the 2NXY-11b-comp_{—}6e_{—}0007 antigen is shown in FIG. 17.
2NXY-11b-redes-8_{—}0105:
Mpmgslqplatlyllgmlvasvlakqvlvnttihfnmwensmvqqmheqiaklkdqqlepcvkltplagatsvit qacpvvswspepikycapqgyailkcnnntfngtgpctnvsevecthgikpvvssqllingslaneevvirsvnftd naktiivqlnssveinctgnghcnitrakwnqtlkqiaqklreqfgenktiifaqssggdpeivthwfncggeffycnst qlfnstwfnstwstegsnntegsdtirlperikqiggyapptsgniscssnitgllltrdggnrnnnseifrpgggdmrd nwrselykykvvsregshhhhhh (SEQ ID NO: 7). A surface representation of the structure of the 2NXY-11b-redes-8_{—}0105 antigen is shown in FIG. 17.
2NXY-11c-25_{—}0188:
Mpmgslqplatlyllgmlvasvlakqplqnvtvdfkmwdndmvddmhdqiakemdeklspcvkltplagats vitqacpktnwnpvpikycapkgfailkcnnatfngtgpctnvstvecthgirpvvssqllingslaetevvirsvnftd naktiivqlntsveinctgdgscniarqkwnqtlqqiaeklrrqfgdnktiifrsssggdpeivthwfncggeffycnstr lfnstwfnstwstegsnntegsqtiqlperikqiggyapptqnhihcssnitgllltrdggnrnndseifrpgggdmrdn wrselykykvvkeegshhhhhh (SEQ ID NO: 8). A surface representation of the structure of the 2NXY-11c-25_{—}0188 antigen is shown in FIG. 17.
2NXY-polar1pt5_{—}0177:
Mpmgslqplatlyllgmlvasvlaktplpnvtqhfdmwnnnmveemhqtiqellkqqltpcvkltplagatsvitq acpkrkwdplpirycappgfailkcnnktfngtgpctnvstvecthgirpvvssqllingslantevvirsvnftdnakt iivqlntsveinctgnghcniarekwnktlkqiakklreqfgsnktiifksssggdpeivthwfncggeffycnstklfn stwfnstwstegsnntegsntielperikqiggyapptedniscssnitgllltrdggnrdnnseifrpgggdmrdnwr selykykvvqregshhhhhh (SEQ ID NO: 9). A surface representation of the structure of the 2NXY-polar1pt5_{—}0177 antigen is shown in FIG. 17.

In some embodiments, a disclosed antigen includes an amino acid sequence at least about 95% identical, such as about 95%, about 96%, about 97%, about 98%, about 99% or even 100% identical to the amino acid sequence according to one of SEQ ID NOs: 1-9. In some embodiments, a disclosed antigen consists of an amino acid sequence according to one of SEQ ID NOs: 1-9.

The disclosed antigens can be produced by the methods described in Section A above. In some embodiments, the disclosed antigen is produced by obtaining the atomic coordinates of a wild-type antigen, wherein a selected monoclonal antibody (such as a neutralizing antibody, for example 2F5, 2G12, b12, or 4E10 in the case of the gp120 antigen from HIV-1) binds the wild-type antigen and amino acids of the wild-type antigen that contact the antibody have been identified.

The amino acids of the wild-type antigen that contact the monoclonal antibody are selected to define the target epitope. The amino acids of the antigen that contact the antibody are selected and maintained in the antigen. In other words, the antigen will include the amino acid residues that contact the antibody.

At least one surface exposed amino acid residue located exterior to the target epitope of the wild-type antigen is selectively mutated. In some embodiments, this includes selectively mutating at least one amino acid on the exterior of the antigen, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 400, 1000, surface exposed amino acids, for example 1-20, 10-30, 20-40, 30-50, 40-60, 50-80, 70-100, 50-200, 100-500, 5-30, 15-45, or 500-100 surface exposed amino acids.

In some examples, between about 10% and about 100% of the surface exposed amino acid residues located exterior to the target epitope are selectively mutated as compared to a wild-type antigen, such as about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%, such as 10% to about 20%, 20% to about 40%, 10% to about 50%, 30% to about 50%, 40% to about 70%, 60% to about 100%, 20% to about 100%, 70% to about 100%, or 20% to about 70% of the surface exposed amino acid residues located exterior to the target epitope are selectively mutated as compared to a wild-type antigen.

In some embodiments, the antigen is glycosylated either naturally or by the introduction of glycosylation sites. In specific examples, the antigen is gp120 and the antigen is glycosylated and includes one or more mutations corresponding to: a) R419N and K421S; b) I420N and Q422S; c) Q422N and I424T; d) I423N and N425T; e) Q246N; f) E267N and E269T; g) K97N and D99T; h) Q103N and H105S; i) N94T; j) Q114N and L116T; k) G222N and A224T; 1) 1201N and Q203T; m) P206N and V208T; n) I423N and N425T; o) M434N and A436S; p) Q442N and R444T; or q) F210N and P212T, in gp120.

The antigens disclosed herein can be chemically synthesized by standard methods, or can be produced recombinantly, for example by expression of the antigen from a nucleic acid molecule that encodes the antigen (see Section C below). An exemplary process for polypeptide production is described in Lu et al., Federation of European Biochemical Societies Letters. 429:31-35, 1998. They can also be isolated by methods including preparative chromatography and immunological separations.

A disclosed antigen can be covalently linked to a carrier, which is an immunogenic macromolecule to which an antigenic molecule can be bound. When bound to a carrier, the bound polypeptide becomes more immunogenic. Carriers are chosen to increase the immunogenicity of the bound molecule and/or to elicit higher titers of antibodies against the carrier which are diagnostically, analytically, and/or therapeutically beneficial. Covalent linking of a molecule to a carrier can confer enhanced immunogenicity and T cell dependence (see Pozsgay et al., PNAS 96:5194-97, 1999; Lee et al., J. Immunol. 116:1711-18, 1976; Dintzis et al., PNAS 73:3671-75, 1976). Useful carriers include polymeric carriers, which can be natural (for example, polysaccharides, polypeptides or proteins from bacteria or viruses), semi-synthetic or synthetic materials containing one or more functional groups to which a reactant moiety can be attached. Bacterial products and viral proteins (such as hepatitis B surface antigen and core antigen) can also be used as carriers, as well as proteins from higher organisms such as keyhole limpet hemocyanin, horseshoe crab hemocyanin, edestin, mammalian serum albumins, and mammalian immunoglobulins. Additional bacterial products for use as carriers include bacterial wall proteins and other products (for example, streptococcal or staphylococcal cell walls and lipopolysaccharide (LPS)).

C. Polynucleotides Encoding Antigens

Polynucleotides encoding the antigens disclosed herein are also provided. These polynucleotides include DNA, cDNA and RNA sequences which encode the antigen.

Methods for the manipulation and insertion of the nucleic acids of this disclosure into vectors are well known in the art (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A nucleic acid encoding an antigen can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding an antigen include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides of the invention can be ribonucleotides, deoxyribonucleotides, or modified forms of either nucleotide. The term includes single and double forms of DNA.

DNA sequences encoding the antigen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding antigens can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell. Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell. Biol., 5:431-437; Sorge et al., 1984, Mol. Cell. Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

In some embodiments, a nucleic acid molecule that encodes a disclosed antigen is a nucleic acid molecule that encodes an antigenically-cloaked gp120 antigen. Exemplary nucleic acid molecules encoding antigenically-cloaked gp120 antigens are given below as SEQ ID NOs: 10-18.

2NXY_{—}11b_{—}1:
Atgcccatgggcagcctgcagcccctggccaccctgtacctgctgggcatgctggtggccagcgtgctggccaccac cgtgctggtgaacgtgaccgtgaccttcgacatgtggaagaacgacatggtggagcagatggacgaggccatcaagac cctgctggacaccagcctgaagccctgcgtgaagctgacccccctggccggcgccaccagcgtgatcacccaggcct gccccaccgtgagctgggagcccatccccatcaggtactgcgccccccccggctacgccatcctgaagtgcaacaaca agaccttcaacggcaccggcccctgcaccaacgtgagcgtggtgacctgcacccacggcatcaggcccgtggtgagc agccagctgctgctgaacggcagcctggccgacgaggaggtggtgatcaggagcgtgaacttcaccgacaacgccaa gaccatcatcgtgcagctgaacaccagcgtggagatcaactgcaccggcgccggccactgcaacatcaccagggcca agtggaacaacaccctgaagcagatcgccgagaagctgagggagcagtteggcaacaacaagaccatcatcttcaag cagagcageggeggcgaccccgagatcgtgacccactggttcaactgeggcggcgagttcttctactgcaacagcacc cagctgttcaacagcacctggttcaacagcacctggagcaccaagggcagcaacaacaccgagggcagcgacaccat caccctgccctgcaggatcaagcagatcggeggctacgccccccccgtgageggcgtgatcacctgcagcagcaaca tcaccggcctgctgctgaccagggacggcggcaacgacaacaacgagagcgagatcttcaggcccggcggcggcg acatgagggacaactggaggagcgagctgtacaagtacaaggtggtgaagctggagggatcccatcatcatcatcatc attag (SEQ ID NO: 10). A surface representation of the structure of the antigen encoded by SEQ ID NO: 10 is shown in FIG. 16.
SIV-8b-sg-11b:
Atgcccatgggcagcctgcagcccctggccaccctgtacctgctgggcatgctggtggccagcgtgctggccaccac cgtgctggtgaacgtgaccgtgaccttcgactggtgcaagaacgacatggtggcccagatgaacaccgccatctgcac cctgtggaagaccagcaacaagccctgcgtgaagctgacccccctgtgcgtgggcgccggcagctgcaacaccagc gtgatcacccaggcctgccccaccgtgagcttcgagcccatccccatcaggtactgcgccccccccggctacgccatc ctgaagtgcaacaacaagaccttcaacggcaccggcccctgcaccaacgtgagcgtggtgacctgcaccgacggcat caggcccgtggtgagcagccagctgctgctgaacggcaccctggccgacgaggaggtggtgatcaggagctgcaact tcaccgacaacgccaagaccatcatcgtgcagctgaacaccagcgtggagatcaactgcaccggcgccggccactgc aacatcaccagggccaagtggaacaacaccctgaagcagatcgccgagaagctgagggagcagttcggcaacaaca agaccatcatcttcaagcagagcagcggcggcgaccccgagatcgtgacccactggttcaactgcggcggcgagttct tctactgcaacagcacccagctgttcaacagcacctggttcaacagcacctggagcaccaagggcagcaacaacaccg agggcagcgacaccatcaccctgccctgcaggatcaagcagateaccggcatgtggtgcaccgtgggcaagatgatgt acgccccccccgtgagcggcgtgatcacctgcagcagcaacatcaccggcctgctgctgaccagggacggcggcaa cgacaacaacgagagcgagatcttcaggcccggcggcggcgacatgagggacaactggaggagcgagctgtacaa gtacaaggtggtgaagctgaccggatcccatcatcatcatcatcattag (SEQ ID NO: 11). A surface representation of the structure of the antigen encoded by SEQ ID NO: 11 is shown in FIG. 16.
SIV_{—}8b_{—}11b_{—}2a:
Atgcctatgggatctctgcagcctctggccacactgtatctgctgggaatgctggtcgcttctgtgctggccacaaccgtg accgtgaatgtgaccgtgaccttcgattggtgcgccgatgatatggtggctacaatgaacaccgccatctgcaccctgtg gaaaaccagcaacgacccctgcaccaagtgtcctaccgtgcggtttaagcccgtgcccatcagatattgtgcccctcctg gctatgccatcctgaagtgcaacaaccgggactttaatggcaccggcccttgcacaaatgtgtccgtggtgacctgtaca gatggcatccaccctgtggtgtctagtcagctgctgctgaatggcacactggccgatgagaaggtggtgatcagaagct gcaacttcagcgacaacgccaagaccatcatcgtgcagctgaacaccagcgtggagatcaattgtacaggccagggcc actgcaatatcacccgggccaagtggaatcagaccctgaagcagatcgccgagaagctgagagagcagttcggcaac aacaagacaatcatcttcaggcctagctctggcggagatcctgagatcgtgacccactggttcaattgcggcggcaagtt cttctactgcaacagcacccagctgttcaacagcacctggttcaactctacttggagcaccaagggcagcaacaacacc gagggcagcgataccatcaccctgccctgcaggatcagatctatcaccggcatggtgtgcacagtgggcaagatgatct acgcccctcctgtggaaggcgtgatcacctgcagcagcaacatcacaggcctgctgctgacaagagatggcggcaac gacaacaacgagagcgagatctttagacctggcggcggagacatgagggacaattggcggagcgagctgtacaagta cagagtggtgcggctgaccggatccggcctgaacgacatcttcgaggcccagaagatcgagtggcacgagctggagg tgctgttccagggcccaggccaccaccaccaccaccactga (SEQ ID NO: 12). A surface representation of the structure of the antigen encoded by SEQ ID NO: 12 is shown in FIG. 16.
SIV_{—}8b_{—}13_{—}2c:
Atgcctatgggatctctgcagcctctggccacactgtatctgctgggaatgctggtcgcttctgtgctggcctctagcgtgt ccgtgaatgtgacccagaccttctcttggtgcgaccaggatatggtggccaaaatgcagcaggccatctgcaatctgtgg caggaaagcgacaccccctgcaatgattgtcccaccaaggcctttagccctcagcctatccagtactgcgcccctaatgg caaggccatcctgaagtgcaacaacgagaacttcaacggcaccggcccttgtacaaatgtgtccgtggtgacctgtaca gccggcattagccctgtggtgtctagtcagctgctgctgaatggcgaactggccgatgagacagtggtgatcagaagct gcaacttcaacgacaacgccaagaccatcatcgtgcagctgaacaccagcgtggagatcaattgtacaggcgagggcc actgcaatatcacccgggccaagtggaatgccaccctgaagcagatcgccaagaagctgagacagcagttcggcaac aacaagacaattatcttccagtcctcttctggcggagatcctgagatcgtgacccactggttcaattgcggcggcagattct tctactgcaacagcacccagctgttcaacagcacctggttcaactctacttggagcaccaagggcagcaacaacaccga gggcagcgatacaatcagcctgccctgccggatcaagagcatcaccgacatgaagtgcagcgtgggcaagatgatcta cgcccctcctaaggccggcgacatcaagtgtagcagcaacatcacaggcctgctgctgacaagagatggcggcaaca ataacaacgagagcgagatctttagacctggcggcggagacatgagggacaattggcggagcgagctgtacaagtac caggtggtggagctgcagggatcccatcatcatcatcatcattag (SEQ ID NO: 13).
NXY-11b-comp-40017:
Atgcctatgggatctctgcagcctctggccacactgtatctgctgggcatgctggtcgcttcagtgctggctcagaaagtg ctggtgaacgtgaccgaggaattcaacatgtggaacaacaacatggtggagctgatgcaccagaagatcgccagcctg atcaaacagagcctgcagccctgtgtgaaactgacacctctggctggcgccacatctgtgatcacccaggcctgtccca aagtggattgggagccccagcctatcgagtattgcgcccctgatggcttcgccatcctgaagtgcaacaacagcaccttc aatggcaccggcccctgtaccaatgtgtctaccgtgcggtgtacacacggcatcagacctgtggtgtctagccagctgct gctgaatggctctctggccagctctgaggtggtgatcagaagcgtgaacttcaccgacaacgccaagaccatcatcgtg cagctgaacaccagcgtggagatcaattgtaccggcgacggcagatgcaatatcgcccgggacaagtggaatgccac actgcagcagatcgcctccaagctgagacagcagttcggcagcaacaagacaatcatcttcgagcagtcctctggcgg agatccagaaatcgtgacccactggttcaactgtggcggcgagttcttctactgcaacagcacccagctgttcaactccac ctggttcaatagcacctggtctactgagggaagcaataacaccgagggctccgataccatcagcctgccctgcagaatc aagcagatcggcggctatgctcctcctaccagaggccagatccggtgcagcagcaatatcacaggcctgctgctgaca agagatggcggcgacagcagcaacgagagcgagatctttagacctggcggcggagacatgagagacaattggcgga gcgagctgtacaagtacaaagtgacccccatcgagggatcccatcatcatcatcatcattag (SEQ ID NO: 14). A surface representation of the structure of the antigen encoded by SEQ ID NO: 14 is shown in FIG. 16.
2NXY-11b-comp_{—}6e_{—}0007:
Atgcctatgggatctctgcagcctctggccacactgtatctgctgggcatgctggtcgcttcagtgctggctagagaagtg ctggtgaacgtgaccgagcagttcaacatgtggcggaaccagatggtggaggccatgcacagagagatcgagcggct ggaaagagccaagctgaacccctgtgtgaaactgacacctctggctggcgccacatctgtgatcacccaggcctgccct aaggtgcagttcgagcccacccctatcacatactgcgcccctgagggctttgccatcctgaagtgcaacaacgacacctt caatggcaccggcccctgtaccaatgtgtccaccgtggactgtacacacggcatcagacccgtgatctccagccagctg ctcctgaatggctctctggccaagggcgaggtggtgatcagaagcgtgaacttcaccgacaacgccaagaccatcatcg tgcagctgaacaccagcgtggagatcaattgcaccggcagaggctactgcaatatcgcccggaagaagtggaacgag acactggaacagatcgccagcaagctgagagatcagttcggcaagaacaagacaatcatcttcagccagtcctctggg ggagatccagaaatcgtgacccactggttcaattgtggcggcgagttcttctactgcaacagcacccagctgttcaacag cacctggttcaactccacctggtctacagaaggaagcaataacaccgagggctccgataccatcaccctgccctgcaga atcaagcagatcggcggctatgctcctcctcagaacggccagatccggtgcagcagcaatatcacaggcctgctgctga caagagatggcggccctagccagaacgagagcgagatctttagacctggcggcggagacatgagagacaattggcg gagcgagctgtacaagtacaaagtgaaggccatcgagggatcccatcatcatcatcatcattag (SEQ ID NO: 15). A surface representation of the structure of the antigen encoded by SEQ ID NO: 15 is shown in FIG. 16.
2NXY-11b-redes-8_{—}0105:
Atgcctatgggatctctgcagcctctggccacactgtatctgctgggcatgctggtcgcttcagtgctggctaaacaggtg ctggtgaacaccaccatccacttcaacatgtgggagaacagcatggtgcagcagatgcacgagcagatcgccaagctg aaggaccagcagctggaaccttgtgtgaagctgacacctctggctggcgccacatctgtgatcacacaggcctgccctg tggtgtcttggagccccgagcctatcaagtattgcgcccctcagggctacgccatcctgaagtgcaacaacaacaccttc aacggcaccggcccctgtacaaatgtgtccgaggtggagtgtacacacggcatcaaaccagtggtctcaagccagctg ctgctgaatggcagcctggccaacgaggaagtggtgatcagaagcgtgaacttcaccgacaacgccaagaccatcatc gtgcagctgaacagcagcgtggagatcaattgcaccggcaacggccactgcaatatcacccgggccaagtggaatca gaccctgaagcagattgcccagaagctgagagagcagttcggcgagaacaagacaatcatcttcgcccagagcagtg gcggagatcctgagatcgtgacccactggttcaactgtggcggcgagttcttctactgcaactctacccagctctttaattc cacatggttcaattccacctggtctacagaaggaagcaataacaccgagggctccgacacaatcagactgccctgccgg atcaagcagatcggaggatacgcccctcctaccagcggcaatatcagctgcagcagcaacatcaccggcctgctgctg acaagagatggcggcaaccggaacaacaacagcgagatcttcagacctggcggcggagacatgagagacaattggc ggagcgagctgtacaagtacaaggtggtgtcccgggagggatcccatcatcatcatcatcattag (SEQ ID NO: 16). A surface representation of the structure of the antigen encoded by SEQ ID NO: 16 is shown in FIG. 17.

### 2NXY-IIc-25_{—}0188:

Atgcctatgggatctctgcagcctctggccacactgtatctgctgggcatgctggtcgcttcagtgctggctaaacagccc ctgcagaacgtgaccgtggacttcaagatgtgggacaacgacatggtggacgacatgcacgaccagatcgccaaaga gatggacgagaagctgtccccttgtgtgaaactgacacctctggctggcgccacatctgtgatcacccaggcctgcccc aagaccaattggaaccccgtgcccatcaagtactgcgcccccaagggctttgccatcctgaagtgcaacaacgccacct ttaatggcaccggcccctgcacaaatgtgtccaccgtggagtgtacacacggcatcagacctgtggtgtctagccagctg ctgctgaatggctctctggccgagacagaggtggtgatcagaagcgtgaacttcaccgacaacgccaagaccatcatcg tgcagctgaacaccagcgtggagatcaattgtaccggcgacggcagctgtaatatcgcccggcagaagtggaatcaga ccctgcagcagatcgccgagaagctgagaaggcagttcggcgacaacaagacaatcatcttcagaagcagctcagga ggagatcctgagatcgtcacccactggttcaactgtggcggcgagttcttctactgcaacagcacccggctgttcaacag cacctggttcaattccacctggtccaccgagggcagcaataatacagagggcagccagaccattcagctcccttgtagg atcaagcagatcggcggctatgcccctcctacccagaaccacatccactgcagcagcaatatcaccggcctgctgctga caagagatggcggcaaccggaacaacgacagcgagatctttagacctggcggcggagacatgagagacaattggcg gagcgagctgtacaagtacaaggtggtgaaagaggaaggatcccatcatcatcatcatcattag (SEQ ID NO: 17). A surface representation of the structure of the antigen encoded by SEQ ID NO: 17 is shown in FIG. 17.
2NXY-polar1pt5_{—}0177:
Atgcctatgggatctctgcagcctctggccacactgtatctgctgggcatgctggtcgctagcgtgctggctaagacccc tctgcctaacgtgacccagcacttcgacatgtggaacaacaacatggtggaggaaatgcaccagaccatccaggaactg ctgaaacagcagctcaccccttgtgtgaaactgacacctctggctggcgccacatctgtgatcacccaggcctgccccaa aagaaaatgggaccccctgcccatcagatattgcgcccctcctggctttgccatcctgaagtgcaacaacaagaccttca atggaaccggaccctgtacaaatgtgtccaccgtggagtgtacacacggcatcagacctgtggtgtctagccagctgctc ctgaatggcagcctggccaataccgaggtggtgatcagaagcgtgaacttcaccgacaacgccaagaccatcatcgtg cagctgaacaccagcgtggagatcaattgcaccggcaacggccactgtaatatcgcccgggagaagtggaataagac cctgaagcagatcgccaagaagctgagagagcagttcggcagcaacaagacaatcatcttcaagagcagcagcggcg gagatccagaaatcgtgacccactggttcaactgtggcggcgagttcttctactgcaacagcaccaagctgttcaacagc acctggttcaattccacctggtccacagagggaagcaataacaccgagggctccaacacaatcgagctgccctgcaga atcaagcagatcggcggctatgcccctcctaccgaggacaacatcagctgcagcagcaacatcacaggcctgctgctg acaagagatggcggcaatagagataacaacagcgagatcttcagacctggaggaggggacatgagagacaattggcg gagcgagctgtacaagtacaaggtggtgcagcgggagggatcccatcatcatcatcatcattag (SEQ ID NO: 18). A surface representation of the structure of the antigen encoded by SEQ ID NO: 18 is shown in FIG. 17.

In some embodiments, a nucleic acid molecule the encodes a disclosed antigen comprises a nucleic acid sequence at least about 95% identical, such as about 95%, about 96%, about 97%, about 98%, about 99% or even 100% identical to the nucleic acid sequence according to one of SEQ ID NOs: 10-18. In some embodiments, a nucleic acid molecule the encodes a disclosed antigen consists of a nucleic acid sequence according to one of SEQ ID NOs: 10-18.

D. Therapeutic Methods and Pharmaceutical Compositions

The antigens as disclosed herein, or a nucleic acid molecule encoding the disclosed antigens, can be administered to a subject in order to generate an immune response to a pathogen.

In exemplary applications, compositions are administered to a subject suffering from a disease, such as disease caused by a pathogen that expresses wild-type antigen or another antigen that contains the target epitope. The antigen is administered in a therapeutically effective amount sufficient to raise an immune response against the pathogen of interest. In some embodiments, the antigen is gp120 antigen, such as an antigenically-cloaked gp120 antigen. The antigenically-cloaked gp120 antigen is administered in an amount sufficient to raise an immune response against HIV virus. Administration induces a sufficient immune response to treat the pathogenic infection, for example, to inhibit the infection and/or reduce the signs and/or symptoms of the infection. Amounts effective for this use will depend upon the severity of the disease, the general state of the subject's health, and the robustness of the subject's immune system. A therapeutically effective amount of the antigen is that which provides either subjective relief of a symptom(s) or an objectively identifiable improvement as noted by the clinician or other qualified observer.

An antigen can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, Pa., 1995) either locally or systemically, such as by intramuscular, subcutaneous, or intravenous injection, but even oral, nasal, or anal administration is contemplated. In one embodiment, administration is by subcutaneous or intramuscular injection. To extend the time during which the disclosed antigen is available to stimulate a response, the antigen can be provided as an implant, an oily injection, or as a particulate system. The particulate system can be a microparticle, a microcapsule, a microsphere, a nanocapsule, or similar particle. (see, e.g., Banga, supra). A particulate carrier based on a synthetic polymer has been shown to act as an adjuvant to enhance the immune response, in addition to providing a controlled release. Aluminum salts can also be used as adjuvants to produce an immune response.

Optionally, one or more cytokines, such as interleukin (IL)-2, IL-6, IL-12, IL-15, RANTES, granulocyte macrophage colony stimulating factor (GM-CSF), tumor necrosis factor (TNF)-α, interferon (IFN)-α or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to the host. In several examples, IL-2, RANTES, GM-CSF, TNF-α, IFN-γ, G-CSF, LFA-3, CD72, B7-1, B7-2, B7-1 B.7-2, OX-40L, 41 BBL and ICAM-1 are administered.

A number of means for inducing cellular responses, both in vitro and in vivo, are known. Lipids have been identified as agents capable of assisting in priming CTL in vivo against various antigens. For example, as described in U.S. Pat. No. 5,662,907, palmitic acid residues can be attached to the alpha and epsilon amino groups of a lysine residue and then linked (for example, via one or more linking residues, such as glycine, glycine-glycine, serine, serine-serine, or the like) to an immunogenic peptide. The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor specific CTL when covalently attached to an appropriate peptide (see, Deres et al., Nature 342:561, 1989).

A pharmaceutical composition including an isolated antigen is provided. In some embodiments, the antigen is mixed with an adjuvant containing two or more of a stabilizing detergent, a micelle-forming agent, and an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54:110, 1977, and Hunter et al., J. Immunol. 129:1244, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

The oil included in the composition is chosen to promote the retention of the antigen in oil-in-water emulsion, i.e., to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse affects, such as granulomas, are evident upon use of the oil.

In one embodiment, the adjuvant is a mixture of stabilizing detergents, micelle-forming agent, and oil available under the name PROVAX® (IDEC Pharmaceuticals, San Diego, Calif.). An adjuvant can also be an immunostimulatory nucleic acid, such as a nucleic acid including a CpG motif, or a biological adjuvant (see above).

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa., 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In another embodiment, a pharmaceutical composition includes a nucleic acid encoding a disclosed antigen. A therapeutically effective amount of the nucleic acid can be administered to a subject in order to generate an immune response. In one specific, non-limiting example, a therapeutically effective amount of a nucleic acid encoding a disclosed antigenically-cloaked gp120 antigen is administered to a subject to treat prostate cancer or breast cancer.

Optionally, one or more cytokines, such as IL-2, IL-6, IL-12, RANTES, GM-CSF, TNF-α, or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically to the host. It should be noted that these molecules can be co-administered via insertion of a nucleic acid encoding the molecules into a vector, for example, a recombinant pox vector (see, for example, U.S. Pat. No. 6,045,802). In various embodiments, the nucleic acid encoding the biological adjuvant can be cloned into same vector as the disclosed antigen coding sequence, or the nucleic acid can be cloned into one or more separate vectors for co-administration. In addition, nonspecific immunomodulating factors such as Bacillus Cahnette-Guerin (BCG) and levamisole can be co-administered.

One approach to administration of nucleic acids is direct immunization with plasmid DNA, such as with a mammalian expression plasmid. As described above, the nucleotide sequence encoding the disclosed antigen can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a disclosed antigen can also be expressed by attenuated viral hosts or vectors or bacterial vectors. Recombinant vaccinia virus, adeno-associated virus (AAV), herpes virus, retrovirus, or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

Simultaneous production of an immunostimulatory molecule and the disclosed antigen enhances the generation of specific effectors. Without being bound by theory, dependent upon the specific immunostimulatory molecules, different mechanisms might be responsible for the enhanced immunogenicity: augmentation of help signal (IL-2), recruitment of professional APC (GM-CSF), increase in CTL frequency (IL-2), effect on antigen processing pathway and MHC expression (IFNγ and TNFα) and the like. For example, IL-2, IL-6, interferon, tumor necrosis factor, or a nucleic acid encoding these molecules, can be administered in conjunction with a disclosed antigen, or a nucleic acid encoding a disclosed antigen. The co-expression of a disclosed antigen together with at least one immunostimulatory molecule can be effective in an animal model to show anti-pathogen effects.

In one embodiment, a nucleic acid encoding a disclosed antigen is introduced directly into cells. For example, the nucleic acid can be loaded onto gold microspheres by standard methods and introduced into the skin by a device such as Bio-Rad's Helios™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 μg/kg to about 50 mg/kg, and typically are about 0.005 mg/kg to about 5 mg/kg (see, e.g., U.S. Pat. No. 5,589,466).

In one specific, non-limiting example, a pharmaceutical composition for intravenous administration would include about 0.1 μg to 10 mg of a disclosed antigen per subject per day. Dosages from 0.1 up to about 100 mg per subject per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

The immunogenic compositions of this disclosure can be employed to generate antibodies that recognize the antigens disclosed herein and the antigen from which the disclosed antigen was derived. The methods include administering to a subject an immunogenic composition including a disclosed antigen or administering to the subject a polynucleotide encoding a disclosed antigens to generate antibodies that recognize the disclosed antigen. The subject employed in this embodiment is one typically employed for antibody production. Mammals, such as, rodents, rabbits, goats, sheep, etc., are preferred.

The antibodies generated can be either polyclonal or monoclonal antibodies. Polyclonal antibodies are raised by injecting (for example subcutaneous or intramuscular injection) antigenic polypeptides into a suitable animal (for example, a mouse or a rabbit). The antibodies are then obtained from blood samples taken from the animal. The techniques used to produce polyclonal antibodies are extensively described in the literature. Polyclonal antibodies produced by the subjects can be further purified, for example, by binding to and elution from a matrix that is bound with the polypeptide against which the antibodies were raised. Those of skill in the art will know of various standard techniques for purification and/or concentration of polyclonal, as well as monoclonal, antibodies. Monoclonal antibodies can also be generated using techniques known in the art.

E. Methods of Inhibiting HIV Infection

Any of the antigenically-cloaked gp120 antigens and nucleic acid molecules encoding the antigenically-cloaked gp120 antigens disclosed herein can be used as immunogens, or to produce immunogens to elicit an immune response (immunogenic compositions) to gp120 such as to a gp120 expressing virus, for example to reduce HIV-1 infection or a symptom of HIV-1 infection. Following administration of a therapeutically effective amount of the disclosed therapeutic compositions, the subject can be monitored for HIV-1 infection, symptoms associated with HIV-1 infection, or both. Disclosed herein are methods of administering the therapeutic molecules disclosed herein (such as antigenically-cloaked gp120 antigens and nucleic acids encoding antigenically-cloaked gp120 antigens) to reduce HIV-1 infection.

Immunogenic compositions can be administered for therapeutic treatments. In therapeutic applications, a therapeutically effective amount of the immunogenic composition is administered to a subject suffering from a disease, such as HIV-1 infection or AIDS.

In therapeutic applications, a therapeutically effective amount of the composition is administered to a subject prior to or following exposure to or infection by HIV. When administered prior to exposure, the therapeutic application can be referred to as a prophylactic administration (such as in the form of a vaccine). Single or multiple administrations of the compositions are administered depending on the dosage and frequency as required and tolerated by the subject. In one embodiment, the dosage is administered once as a bolus, but in another embodiment can be applied periodically until a therapeutic result, such as a protective immune response, is achieved. Generally, the dose is sufficient to treat or ameliorate symptoms or signs of disease without producing unacceptable toxicity to the subject. Systemic or local administration can be utilized.

It may be advantageous to administer the immunogenic compositions disclosed herein with other agents such as proteins, peptides, antibodies, and other antiviral agents, such as anti-HIV agents. Examples of such anti-HIV therapeutic agents include nucleoside reverse transcriptase inhibitors, such as abacavir, AZT, didanosine, emtricitabine, lamivudine, stavudine, tenofovir, zalcitabine, zidovudine, and the like, non-nucleoside reverse transcriptase inhibitors, such as delavirdine, efavirenz, nevirapine, protease inhibitors such as amprenavir, atazanavir, indinavir, lopinavir, nelfinavir, osamprenavir, ritonavir, saquinavir, tipranavir, and the like, and fusion protein inhibitors such as enfuvirtide and the like. In certain embodiments, immunogenic compositions are administered concurrently with other anti-HIV therapeutic agents. In some examples, the disclosed antigens are administered with T-helper cells, such as exogenous T-helper cells. Exemplary methods for the producing and administering T-helper cells can be found in International Patent Publication WO 03/020904, which is incorporated herein by reference.

In certain embodiments, the immunogenic compositions are administered sequentially with other anti-HIV therapeutic agents, such as before or after the other agent. One of ordinary skill in the art would know that sequential administration can mean immediately following or after an appropriate period of time, such as hours, days, weeks, months, or even years later.

The antigenically-cloaked gp120 antigens and nucleic acids encoding these antigenically-cloaked gp120 antigens can be used in a multistep immunization regime. In some examples, the regime includes administering to a subject a therapeutically effective amount of a first antigenically-cloaked gp120 antigen as disclosed herein (the prime) and boosting the immunogenic response with one or more additional antigenically-cloaked gp120 antigens in which the selected antigenic surface is maintained, but the mutations made in the surface exposed non-contact residues are different after an appropriate period of time. The method of eliciting such an immune reaction is what is known as “prime-boost.” In this method, the antibody response to the selected immunogenic surface is focused by giving the subject's immune system a chance to “see” the antigenic surface in multiple contexts. In other words, the use of multiple antigenically-cloaked gp120 antigens with only one antigenic surface in common selects for antibodies that bind the antigen's surface in common. Different dosages can be used in a series of sequential inoculations. Thus, a practitioner may administer a relatively large dose in a primary inoculation and then boost with relatively smaller doses of the boost. The immune response against the antigenically-cloaked gp120 antigen can be generated by one or more inoculations of a subject with a disclosed immunogenic composition.

In some examples, the antigenically-cloaked gp120 antigens and nucleic acids encoding these antigenically-cloaked gp120 antigens can are administered in “prime-boost” immunization regimes with stabilized gp140 trimer (see for example Yang et al. J. Virol. 76(9):4634-42, 2002), and/or stabilized gp120 polypetides (such as those described in WO 07/030,518). In some examples of this method, antigenically-cloaked gp120 antigen is initially administered to a subject and at periodic times thereafter stabilized gp140 trimer boosts are administered. In other examples of this method stabilized gp140 trimer is initially administered to a subject and at periodic times thereafter one or more antigenically-cloaked gp120 antigens are administered. Examples of stabilized gp140 or gp120 trimers can be found for example in U.S. Pat. No. 6,911,205 which is incorporated herein in its entirety.

One can also use cocktails containing a variety of different HIV strains to prime and boost with trimers from a variety of different HIV strains or with trimers that are a mixture of multiple HIV strains. For example, the first prime could be with a gp120 polypeptide from one primary HIV isolate, with subsequent boosts using trimers from different primary isolates.

In one embodiment, a suitable immunization regimen includes at least three separate inoculations with one or more immunogenic compositions of the invention, with a second inoculation being administered more than about two, about three to eight, or about four, weeks following the first inoculation. Generally, the third inoculation is administered several months after the second inoculation, and in specific embodiments, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of HIV-1 infection or progression to AIDS, improvement in disease state (e.g., reduction in viral load), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, the dose of the chimeric non-HIV polypeptide or polynucleotide and/or adjuvant can be increased or the route of administration can be changed.

It is contemplated that there can be several boosts, and that each boost can be a different antigenically-cloaked antigen, so long as the selected antigenic surface is the same. It is also contemplated that in some examples that the boost may be the same disclosed antigen as another boost, or the prime.

The prime can be administered as a single dose or multiple doses, for example two doses, three doses, four doses, five doses, six doses or more can be administered to a subject over days, weeks or months. The boost can be administered as a single dose or multiple doses, for example two to six doses, or more can be administered to a subject over a day, a week or months. Multiple boosts can also be given, such one to five, or more. Different dosages can be used in a series of sequential inoculations. For example a relatively large dose in a primary inoculation and then a boost with relatively smaller doses. The immune response against the selected antigenic surface can be generated by one or more inoculations of a subject with an immunogenic composition disclosed herein.

F. Immunodiagnostic Reagents and Kits

In addition to the therapeutic methods provided above, any of the disclosed antigens disclosed herein can be utilized to produce antigen specific immunodiagnostic reagents, for example, for serosurveillance. Immunodiagnostic reagents can be designed from any of the antigenic polypeptide described herein. For example, in the case of the antigenically-cloaked gp120 antigens, the presence of serum antibodies to HIV is monitored using the isolated cloaked gp120 antigens disclosed herein, such as to detect an HIV infection and/or the presence of antibodies that specifically bind the focused antigenic surface of the cloaked gp120 antigens.

Generally, the method includes contacting a sample from a subject, such as, but not limited to a blood, serum, plasma, urine or sputum sample from the subject with one or more of the disclosed antigens disclosed herein (including a polymeric form thereof) and detecting binding of antibodies in the sample to the disclosed antigens. The binding can be detected by any means known to one of skill in the art, including the use of labeled secondary antibodies that specifically bind the antibodies from the sample. Labels include radiolabels, enzymatic labels, and fluorescent labels.

Any such immunodiagnostic reagents can be provided as components of a kit. Optionally, such a kit includes additional components including packaging, instructions and various other reagents, such as buffers, substrates, antibodies or ligands, such as control antibodies or ligands, and detection reagents

Methods are further provided for a diagnostic assay to monitor HIV-1 induced disease in a subject and/or to monitor the response of the subject to immunization by an HIV vaccine. By “HIV-1 induced disease” is intended any disease caused, directly or indirectly, by HIV. An example of an HIV-1 induced disease is acquired autoimmunodeficiency syndrome (AIDS). The method includes contacting a disclosed antigenically-cloaked gp120 antigen with a sample of bodily fluid from the subject, and detecting binding of antibodies in the sample to the disclosed antigens. In addition, the detection of the HIV-1 binding antibody also allows the response of the subject to immunization by a HIV vaccine to be monitored. In still other embodiments, the titer of the HIV-1 binding antibodies is determined. The binding can be detected by any means known to one of skill in the art, including the use of labeled secondary antibodies that specifically bind the antibodies from the sample. Labels include radiolabels, enzymatic labels, and fluorescent labels. In other embodiments, a disclosed antigenically-cloaked gp120 antigens is used to isolate antibodies present in a subject or biological sample obtained from a subject.

G. Exemplary Computer System

FIG. 14 illustrates an exemplary computer system 120 that can serve as an operating environment for the software for the computational design of an antigen and storing data related to the antigen, including the atomic coordinates of the antigen, for example to carry out the computational design of the disclosed antigens. In some examples, the atomic coordinates are obtained from an antigen of interest, for example by downloading the atomic coordinates from the PDB. Examples of atomic coordinates the can be used with the disclosed methods include the atomic coordinates of gp120 in complex with an antibody, such as those described in WO 07/030,518, incorporated herein by reference.

With reference to FIG. 14 an exemplary computer system for implementing the disclosed method includes a computer 120 (such as a personal computer, laptop, palmtop, set-top, server, mainframe, hand held device, and other varieties of computer), including a processing unit 121, a system memory 122, and a system bus 123 that couples various system components including the system memory to the processing unit 121. The processing unit can be any of various commercially available processors, including INTEL® x86, PENTIUM® and compatible microprocessors from INTEL® and others, including Cyrix, AMD and Nexgen; Alpha from Digital; MIPS from MIPS Technology, NEC, IDT®, Siemens, and others; and the PowerPC from IBM® and Motorola. Dual microprocessors and other multi-processor architectures also can be used as the processing unit 121.

The system bus can be any of several types of bus structure including a memory bus or memory controller, a peripheral bus, and a local bus using any of a variety of conventional bus architectures such as PCI, VESA, AGP, Microchannel, ISA and EISA, to name a few. A basic input/output system (BIOS), containing the basic routines that help to transfer information between elements within the computer 120, such as during start-up, is stored in ROM 124. The system memory includes read only memory (ROM) 124 and random access memory (RAM) 125.

The computer 120 may further include a hard disk drive 127, a magnetic disk drive 128, for example to read from or write to a removable disk 129, and an optical disk drive 130, for example to read a CD-ROM disk 131 or to read from or write to other optical media. The hard disk drive 127, magnetic disk drive 128, and optical disk drive 130 are connected to the system bus 123 by a hard disk drive interface 132, a magnetic disk drive interface 133, and an optical drive interface 134, respectively. The drives and their associated computer readable media provide nonvolatile storage of data, data structures (databases), computer executable instructions, etc. for the computer 120. Although the description of computer readable media above refers to a hard disk, a removable magnetic disk and a CD, it should be appreciated by those skilled in the art that other types of media which are readable by a computer, such as magnetic cassettes, flash memory cards, digital video disks, Bernoulli cartridges, and the like, can also be used in the exemplary operating environment.

Data representative of the atomic structure of a protein or peptide can be stored in the drives and RAM 125, including an operating system 135, one or more application programs 136, other program modules 137, and program data 138.

A user can enter commands and information into the computer 120 using various input devices, such as a keyboard 140 and pointing device, such as a mouse 142. Other input devices (not shown) can include a microphone, satellite dish, scanner, or the like. These and other input devices are often connected to the processing unit 121 through a serial port interface 146 that is coupled to the system bus, but can be connected by other interfaces, such as a parallel port, game port or a universal serial bus (USB). A monitor 147 or other type of display device is also connected to the system bus 123 via an interface, such as a video adapter 148. In addition to the monitor, computers typically include other peripheral output devices (not shown), such as printers. In some examples, data is collected from a physical object, such as an antigen and transformed and displayed in a medium that can be perceived by the user, for example on a monitor or other device. Alternatively, the display medium is a print-out or other tangible medium. The output information can also be preserved in a computer readable medium for storage and/or subsequent use or display.

The computer 120 can operate in a networked environment using logical connections to one or more other computer systems, such as computer 102. The other computer systems can be servers, routers, peer devices or other common network nodes, and typically include many or all of the elements described relative to the computer 120, although only a memory storage device 149 has been illustrated in FIG. 14. The logical connections depicted in FIG. 14 include a local area network (LAN) 151 and a wide area network (WAN) 152. Such networking environments are common in offices, enterprise-wide computer networks, intranets and the Internet.

When used in a LAN networking environment, the computer 120 is connected to the local network 151 through a network interface or adapter 153. When used in a WAN networking environment, the computer 120 typically includes a modem 154 or other means for establishing communications (for example via the LAN 151 and a gateway or proxy server 155) over the wide area network 152, such as the Internet. The modem 154, which can be internal or external, is connected to the system bus 123 via the serial port interface 146. In a networked environment, program modules depicted relative to the computer 120, or portions thereof, can be stored in the remote memory storage device. It will be appreciated that the network connections shown are exemplary and other means of establishing a communications link between the computer systems (including an Ethernet card, ISDN terminal adapter, ADSL modem, 10BaseT adapter, 100BaseT adapter, ATM adapter, or the like) can be used.

The methods, including the acts and operations they comprise, described above can be performed by the computer 120 or by an instrument or other device that is specifically programmed or dedicated to perform the disclosed methods. Hence, the methods can be carried out on a specific machine, such as a device other than a general purpose computer. Such acts and operations are sometimes referred to as being computer executed. It will be appreciated that the acts and symbolically represented operations include the manipulation by the processing unit 121 of electrical signals representing data bits which causes a resulting transformation or reduction of the electrical signal representation, and the maintenance of data bits at memory locations in the memory system (including the system memory 122, hard drive 127, floppy disks 129, and CD-ROM 131) to thereby reconfigure or otherwise alter the computer system's operation, as well as other processing of signals. The memory locations where data bits are maintained are physical locations that have particular electrical, magnetic, or optical properties corresponding to the data bits.

H. Exemplary Distributed Computing Environment

FIG. 15 illustrates a distributed computing environment in which the software and/or database elements used to implement the methods of the present disclosure may reside. The distributed computing environment 200 includes two computer systems 202, 204 connected by a connection medium 206, although the disclosed method is equally applicable to an arbitrary, larger number of computer systems connected by the connection medium 206. The computer systems 202, 204 can be any of several types of computer system configurations, including personal computers, multiprocessor systems, handheld devices, and the like. In terms of logical relation with other computer systems, a computer system can be a client, a server, a router, a peer device, or other common network node. Additional computer systems 202 or 204 may be connected by an arbitrary number of connection mediums 206. The connection medium 206 can comprise any local area network (LAN), wide area network (WAN), or other computer network, including but not limited to Ethernets, enterprise-wide computer networks, intranets and the Internet.

Portions of the software for computationally designing antigens and databases for storing data can be implemented in a single computer system 202 or 204, with the application later distributed to other computer systems 202, 204 in the distributed computing environment 200. Portions of the software may also be practiced in a distributed computing environment 200 where tasks are performed by a single computer system 202 or 204 acting as a remote processing device that is accessed through a communications network, with the distributed application later distributed to other computer systems in the distributed computing environment 200. In a networked environment, program modules comprising the software for computationally designing antigens and databases for storing data can be located on more than one computer system 202 or 204. Communication between the computer systems in the distributed computing network may advantageously include encryption of the communicated data.

### EXAMPLES

### Example 1

This example describes the design of antigenically cloaked gp120 antigen.

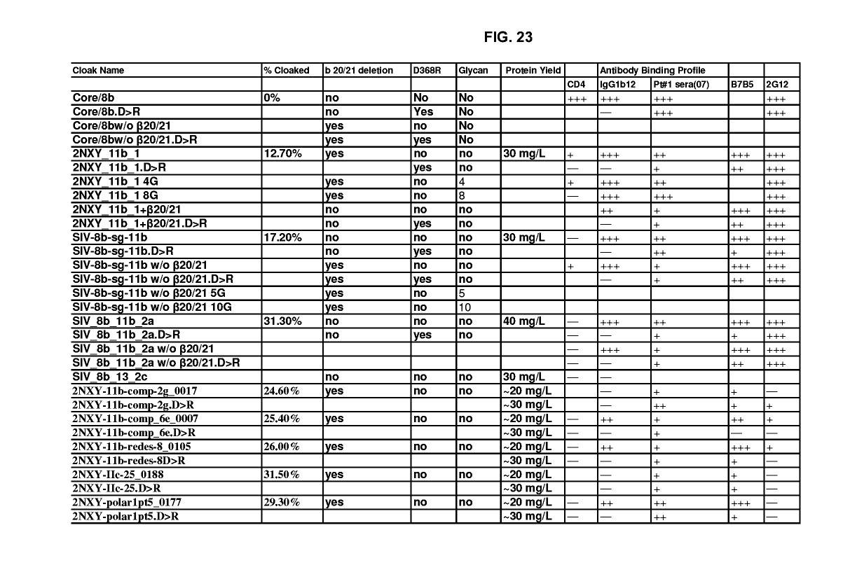

While this example focuses primarily on the cloaking of gp120 based on the PDB structure 2NY7 which contains the complex of b12 with gp120 (available in the PDB at Accession No. 2NY7; the 2NY7 structure which is incorporated herein in its entirety), the design parameters are equally applicably to other antigens, such as other antigenically cloaked gp120 antigens. The wild-type gp120 antigen used as a starting point for antigenic cloaking was a mutant version that has been stabilized by space-filling mutations and addition of three disulfide bonds (“DS12 F123” see Zhou et al. Nature 2007, 445, 732-737 incorporated by reference herein in it entirety). The cloaks based on 2NY7 are the “SIV_{—}8B” cloaks, see the Table shown in FIG. 23. Additional antigenically cloaked gp120 antigen cloaks discussed in this application were based on the atomic structure of the complex of CD4 with gp120 available in the PDB at Accession No. 2NXY; the 2NY7 structure which is incorporated herein in its entirety. The 2NXY cloaks were designed in a similar fashion to the 2NY7 cloaks, but in the case of the 2NXY cloaks, special use was made of the crystal structure of the non-neutralizing antibody F105 in complex with gp120, to assist in the design of specific mutations to knock-out F105 binding. The 2NXY cloaks and the F105-killing mutations are discussed at the end of this Example.

Antigenically cloaked gp120 antigen was computationally designed in an iterative manner, with feedback from structural analysis of designed cloaked proteins, and in some examples iterations due to feedback from experimental testing of designed antigenically cloaked gp120 antigen. The ROSETTADESIGN program was used for computational protein design.

First, initial cloaking positions on the antigen—surface positions to be designed—were selected. As discussed below, not all of these positions are mutated in the final constructs chosen for testing. Cloaking positions on the antigen were surface exposed residues excluding (a) residues that contact the antibody (b) N-glycosylation sequons (NXS/T, where X is any residue except Proline), and (c) residues contacting any N-acetyl-glucosamine (NAG) groups attached to Asn at glycosylation sites. Surface exposed residues were defined using the program NACCESS (Hubbard, S. J. & Thornton, J. M. (1993), ‘NACCESS’, Computer Program, Department of Biochemistry and Molecular Biology, University College London) as residues with >40% sidechain surface area exposed, relative to the same sidechain in an isolated tripeptide. Contact residues—residues on the antigen gp120 that contact the antibody b12—were defined broadly (to avoid altering b12-epitope) as any antigen residue with at least one heavy-atom (non-hydrogen atom) within 8.0 angstroms of a heavy-atom on the antibody. In later generations of cloaked molecules, the definition of contact residues could be narrowed. N-glycosylation sequences were identified by sequence analysis. Residues contacting NAG groups were defined as any residue with at least one sidechain heavy atom within 6.0 angstroms of any heavy atom on the NAG group.

With the cloaking positions defined, the amino acids allowed at each position were assigned either automatically or semi-automatically, using different cases to test different strategies. In some cases all amino acids, or only polar and charged amino acids, were allowed at each position (“de novo cloaking”); in other cases the allowed amino acids at each position were restricted to amino acids occurring at that position in a multiple-sequence alignment of gp120 from SIV and HIV2 homologs (“evolutionary cloaking”); in other cases the allowed amino acids at each position were restricted to amino acids from homologs as above, but amino acids from a HIV1 multiple sequence alignment were also allowed; and in other cases the various strategies were mixed, in an effort to generate greater antigenic diversity while maintaining stability and solubility. Another variance between cases was the degree to which non-design positions were allowed to repack. In some cases all non-design positions were fixed in their native rotamers, and in other cases non-design surface positions were allowed to repack if they were neighboring design positions.

Restrictions were implemented at various design positions in many cases, often based on analysis of results of cases already tested. The restrictions were aimed at (a) improving solubility by excluding large hydrophobic residues and/or forcing the inclusion of polar and charged amino acids at highly exposed positions, (b) maintaining stability by preventing mutations in surface positions that were particularly well-packed or were making apparently important hydrogen bonds in the native protein, where ROSETTADESIGN failed to generate similarly well-packed mutations—this most often occurred on the surface of beta-sheets, (c) optimizing alpha-helical propensities by limiting use of short polars or beta-branched amino acids at exposed positions on helices, in cases where ROSETTADESIGN favored such amino acids.

Once the design positions and the ‘library’ of amino acids at each position were initially defined, automated protein design was carried out using ROSETTADESIGN (Kuhlman et al. (2003) Science, 302, 1364-1368; Kuhlman et al. Proc Natl Acad Sci USA. 2000 Sep. 12; 97(19):10383-8). ROSETTADESIGN uses an all-atom energy function to score amino acid sequences on a fixed protein backbone, and a Monte Carlo method to rapidly sample sequence space.

The ROSETTADESIGN energy function is a linear combination of a Lennard-Jones 6-12 potential, an empirical, geometry-based hydrogen-bonding potential (Kortemme et al. (2003) J. Mol. Biol. 326, 1239-1259), the Lazaridis-Karplus implicit solvation model (Lazaridis, T. & Karplus, M. (1999) Proteins: Struct. Funct. Genet. 35, 133-152), backbone-dependent rotamer probabilities (Dunbrack & Cohen (1997). Protein Sci. 6, 1661-1681), amino acid probabilities as a function of phi/psi backbone angles, an empirical electrostatics-motivated term derived from the distance distributions between polar residues in high-resolution crystal structures (Simons et al (1999) Proteins: Struct. Funct. Genet. 34, 82-95), and reference energies for each amino acid that reflect their frequency of occurrence in protein structures. Weights for the different energy terms are determined as described (Saunders & Baker (2002) J. Mol. Biol. 322, 891-90).

Multiple variants of the standard ROSETTADESIGN energy function may be employed, with additional terms, modified terms, and/or modified weight sets. In this work we used both the standard energy function and a modified version that was recently parameterized to best reproduce native sequences when redesigning whole proteins in fixed backbone simulations (command line option, -soft_rep_design, ROSETTADESIGN version 2.1). This variant of the energy function has a modified “softened” repulsive component of the 6-12 Lennard Jones potential and slightly increased atomic radii that combine to better reproduce atom-atom distance distributions compared to the standard energy function in rotamer recovery tests on native proteins (Dantas et al. JMB 2007, 366, 1209-1221). This version of the energy function, referred to as “Rosetta_DampRep” has proven effective in a protocol to identify mutations at protein-protein interfaces that increase binding affinity (Sammond et al. JMB 2008, 371,1392)

Computational design of gp120 cloaks was carried out in an iterative fashion, with the goal of converging on a highly cloaked molecule that maintained stability, solubility and target antibody binding of the native gp120 protein. There were two rounds of computational design, each followed by a round of experimental characterization. In each round of automated design, multiple cases were tested as mentioned above. For each case, three hundred designs were generated, and that typically translated into 1-20 unique designs with unique sequences and energies, though the sequences typically differed in only few positions. These were ranked by energy and underwent human structural analysis and case-comparison.

Thirteen different cases were tested for 2NY7-based cloaks in the first round of computational design. One 2NY7-based cloak was initially chosen for experimental testing: “siv_{—}8b_sg_{—}11b.” In this case, the initial amino acids at each design position were selected from SIV homologs, but this was modified to (a) exclude hydrophobics at nearly all surface positions except on beta-sheets, (b) include all polars at every design position, and (c) allow the native amino acid from HIV1 HXB2 strain in case the native sidechain has highly optimal packing—and (c) superceeded (a). Also, in this case, non-design positions were held fixed in their native rotamers.

Following experimental feedback on the initial cloaked molecule, several additional cases were tested computationally in attempts to increase the fraction of the molecular surface that was cloaked on “siv_{—}8b_sg_{—}11b” while maintaining stability, solubility, and target antibody binding. The first round cloak “siv_{—}8b_sg_{—}11b” retained—nM b12 affinity with 38 surface mutations and 17% of the total surface area of gp120 modified ('cloaked') compared to the gp120 in 2NY7 (without b12 present). In the example of the cloak “siv_{—}8b_{—}11b_{—}2a,” which derived from the first round cloak “siv_{—}8b_sg_{—}11b,” an additional 21 design positions were selected based on both exposure and distance from first-round mutations. The goal was to ensure that as many potential antibody footprints in an area of ˜20 Å² outside the b12-binding site contained at least one mutation. Some (seven) of the new design positions had been allowed as design positions in the first round, but ROSETTADESIGN had chosen to keep the native amino acid which was allowed in the case of siv_{—}8b_sg_{—}11b as noted above. Eight other new design positions were allowed even though the native amino acid was near a NAG, and six other new design positions were allowed even though the native amino acid sidechain was slightly less than 40% exposed. In this second round, the native amino acid was not allowed at the new design positions—maximizing increased cloaking surface coverage. Also in the second round, all first round design positions were allowed to repack, to optimize design at the new design positions, because some of the new design positions were adjacent to first round positions. As in the first round, designs were ranked by energy and the best unique sequences were structurally analyzed. In this case the lowest energy design was chosen without further modification for further experimental testing—this was the cloak “siv_{—}8b_{—}11b_{—}2a.”

An additional cloak was designed in the second round—siv_{—}8b_{—}13_{—}2c. This second cloak was designed with the intent of being antigenically different from siv_{—}8b_sg_{—}11b and its derivative siv_{—}8b_{—}11b_{—}2a. Toward that end, a two-step process was carried out. First, the same set of amino acids was allowed at all design positions as in the case of siv_{—}8b_sg_{—}11b, except that, at most positions, the particular amino acids used in the siv_{—}8b_sg_{—}11b design were disallowed. The best design by energy from that step was carried forward to the second stage, in which the same additional design positions were allowed as for siv_{—}8b_{—}11b_{—}2a, but at each design position the amino acid chosen for siv_{—}8b_{—}11b_{—}2a was disallowed. This process resulted in unfavorable choices at several positions where all the allowed amino acids produced clashes—in those cases compromises were made to avoid clashes at the expense of reduced antigenic diversity.

Similar efforts were made to generate increased antigenic diversity in the series of second round 2NXY cloaks. Briefly: The cloak “2NXY-polar1pt5_{—}0177” was generated using an expanded set of design positions and nearly only polars were allowed at all design positions. The cloak “2NXY-11c-25_{—}0188” was generated using the same design positions as “2NXY-polar1pt5_{—}0177,” but the amino acids chosen for “2NXY-polar1pt5_{—}0177” were disallowed at most design positions, and native amino acids were disfavored directly by assigning them a small energetic penalty. The cloaks “2NXY-11b-comp-2g” and “2NXY-11b-comp-6e” were designed to utilize different cloaking positions compared to the first round 2NXY-11b cloak and compared to the “2NXY-polar1pt5_{—}0177” and “2NXY-11c-25_{—}0188” wherever possible. These were also polar cloaks, and the “6e” version was designed to be antigenically different than the “2g” cloak by disallowing amino acids chosen for the “2g” cloak. The “2NXY-11b-redes-8” cloak used the same design positions as the first round cloak 2NXY-11b, but expanded beyond those positions, and disallowed the amino acids used in the first round cloak. As assessed by fraction of surface area mutated, all of these second round 2NXY cloaks were substantially different from each other and from the 8b cloaks.

The 2NXY cloaks were also designed with mutations at key contact locations for the antibody F105, with the goal of eliminating F105-binding. The atomic coordinates of the F105-binding patch were defined by a crystal structure of the F105 antibody and gp120. Mutations were designed to either remove key contacts or introduce clashes or both. To maintain a diverse antigenic surface outside the b12-binding site, different sets of F105-killing mutations were introduced in different cloaks, to as great as degree as was possible.

Finally, the 2NXY cloaks contained trimmed V1/V2 and β20/21 regions to reduce the immunogenicity or at least alter the antigenicity of those regions. The modified V1/V2 modification was taken from a core gp120 previously designed in that had improved expression yields known as “new 9c”, and that modification was to use the following sequence between C119 and C205: VKLTPLAGATSVITQA A (SEQ ID NO: 24). The β20/21 was trimmed on most of the 2NXY cloaks based on rational design, and the modification was to use the following sequence between I423 and Y435: GG. Though these modifications were not done using computational design, they do serve as examples of cloaking by modifying the backbone of the protein rather than just the sequence. Computational methods for flexible backbone protein design allow this more aggressive method of cloaking to be applied to loop-trimming as in the case of V1/V2 and β20/21, but also to trimming, modifying, or even building new backbone in more complex structural contexts.

### Example 2

This example describes the production of cloaked gp120 antigens and immunization of rabbits with the cloaked gp120 antigens.

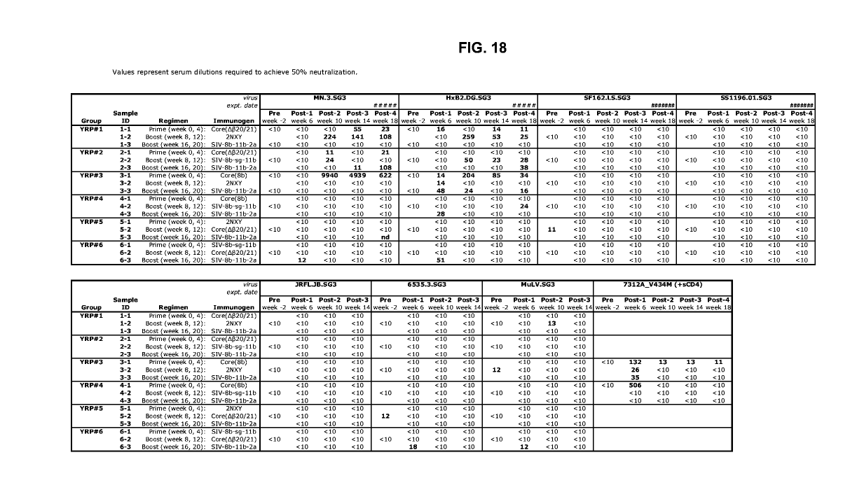

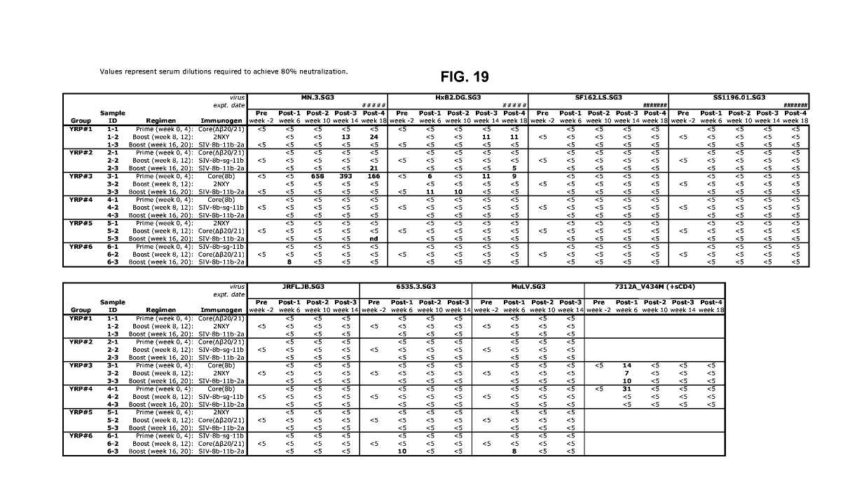

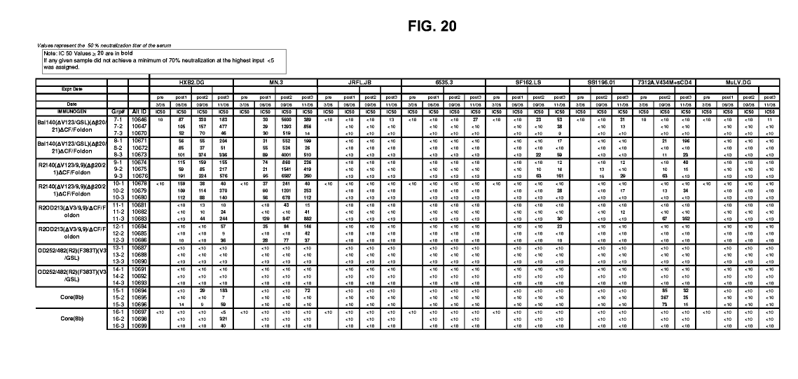

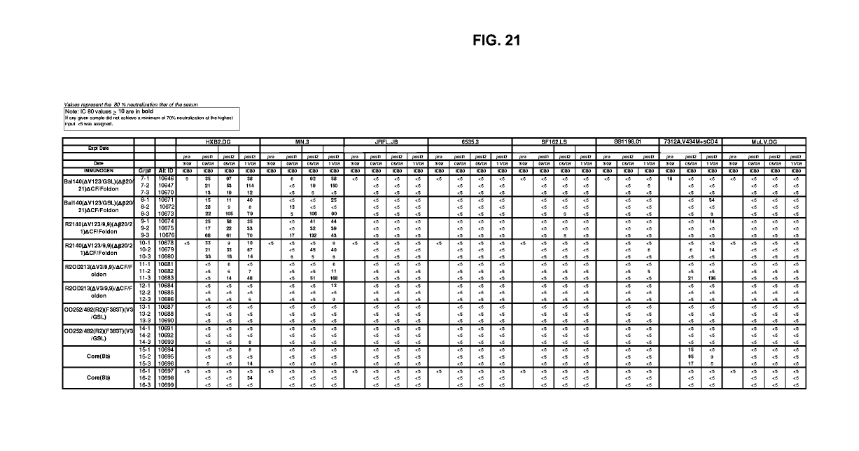

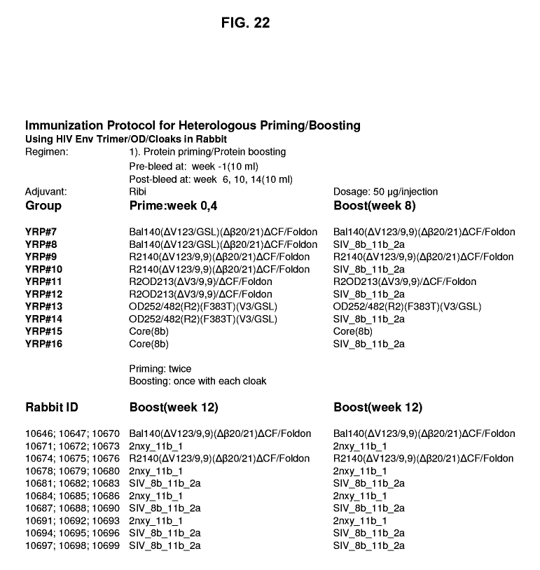

Genes encoding each antigen listed in FIGS. 17-23 were cloned into expression vector CMV/R. Expression vectors were then transfected into 293F cells using 293Fectin (Invitrogen, Carlsbad, Calif.). Five days after transfection, cell culture supernatant was harvested and concentrated/buffer-exchanged to 500 mM NaCl/50 mM Tris pH8.0. The protein initially was purified using HiTrap IMAC HP Column (GE, Piscataway, N.J.), and subsequent gel-filtration using Superdex™200 (GE). Finally, the His tag was cleaved off using 3C protease (Novagen, Madison, Wis.).

For vaccinations with the antigens listed in FIGS. 17-23, 3-4 months old rabbits (NZW)(Covance, Princeton, N.J.) were immunized using the indicated antigens with Sigma Adjuvant System (Sigma, St. Louis, Mo.) according to manufacture's protocol. Specifically, three rabbits in each group were vaccinated with 50 μg of protein in 300 μl PBS emulsified with 300 μl of adjuvant intramuscularly (both legs, 300 μl each leg) for example at week 0, 4, 8, 12, 16. Sera were collected for example at week 6 (Post-1), 10 (Post-2), 14 (Post-3), and 18 (Post-4), and subsequently analyzed for their neutralization activities against a panel of HIV-1 strains, and the profile of antibodies that mediate the neutralization (see Tables in FIGS. 17 through 23).

The cloaked antigens were also tested for antigenic profiling using well-characterized human monoclonal antibodies. With specific reference to the SIV_{—}8b_{—}11b_{—}2a antigen, four categories of monoclonal antibodies were used to probe the antigenicity of SIV_{—}8b_{—}11b_{—}2a:

1) potent neutralizing CD4 binding site (CD4BS) antibody (IgG₁b12, the b12 neutralizing antibody);

2) the non-neutralizing CD4 binding site antibodies (IgG₁b13 and F105);

3) the non-neutralizing CD4 induced (CD41) antibodies (17b, 48d, m6); and

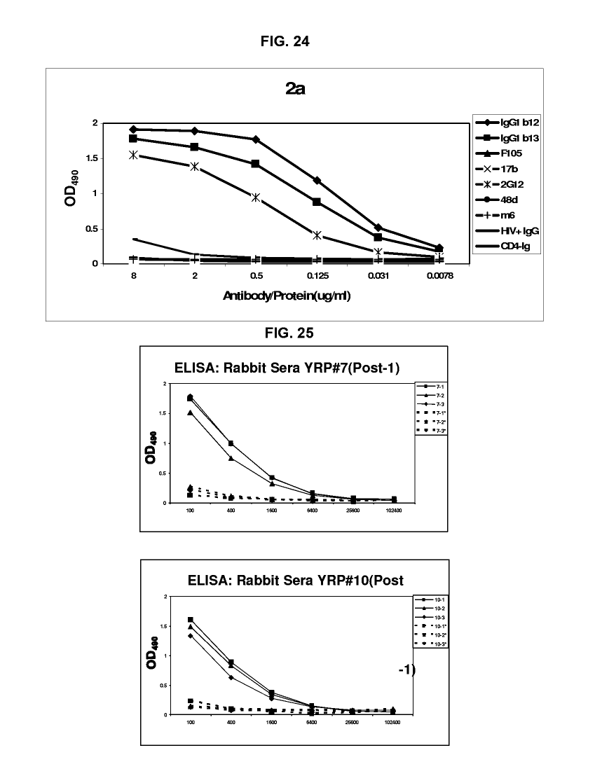

4) the broad neutralizing antibody 2G12 which binds to glycan motifs on the outer domain, serving as a control for structural integrity of the cloak protein (see FIG. 24).

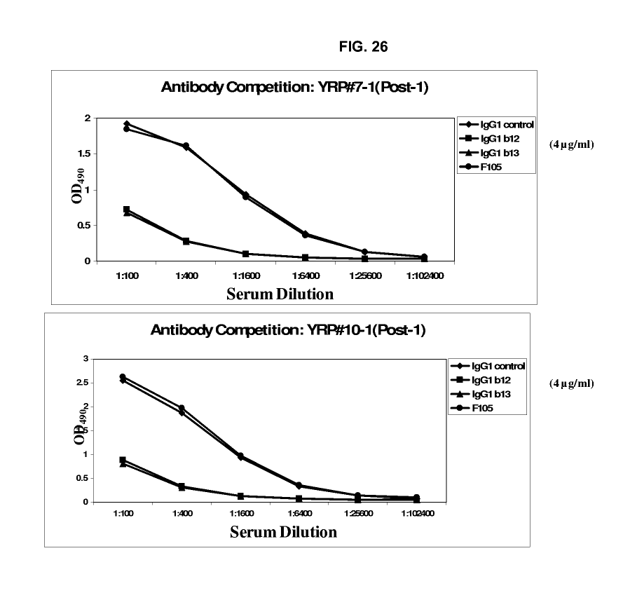

In some trials, cloaked antigen was used to coat ELISA plates (4 μg/ml in PBS). An amount of the different antibodies as indicated was added to the wells, and incubated at room temperature for one hour. The plates were washed six times with PBS+0.05% Tween20, and followed by incubation with HRP conjugated goat anti-human IgG (1:5000) for another hour. The plates were washed again six times with PBS+0.05% Tween20, and developed by adding OPN substrate for 30 minutes at room temperature. The results were evaluated by plotting the readout at OD490 vs. the antibody concentrations. As shown in FIG. 25, SIV_{—}8b_{—}11b_{—}2a retained its binding capacity to IgG₁b12, IgG₁b13, and 2G12, while it lost most of its binding to non-neutralizing CD4BS and CD41 antibodies (FIG. 26). Results of trials with other cloaked antigens area shown in the Table that is depicted in FIG. 23. Further modification of SIV_{—}8b_{—}11b_{—}2a at position 369(P369R) eliminated the binding of non-neutralizing CD4 BS IgG₁b13.

The cloaked antigens were also used to probe for rabbit anti-sera for existence of CD4BS antibodies in the anti-sera. With specific reference to SIV_{—}8b_{—}11b_{—}2a, this antigen was used to probe rabbit anti-sera for existence of CD4BS antibodies in the anti-sera (see FIG. 25). As a negative control SIV_{—}8b_{—}11b_{—}2a(Δ371I) was created, which lost binding to IgG₁b12 through deletion of amino acid 3711, but retained its binding to the antibody 2G12 at normal level.

Anti-sera #7 and #10 were generated by vaccination of rabbits using trimeric HIV-1 envelope protein. Both anti-sera showed differential binding toward SIV_{—}8b_{—}11b_{—}2a and control SIV_{—}8b_{—}11b_{—}2a(Δ371I), indicating the existence of CD4 BS antibodies in both anti-sera tested. Those binding can be competed by IgG₁b12, further confirming this result.

### Example 3

### Treatment of HIV in a Subject

This example describes exemplary methods for treating or inhibiting an HIV infection in a subject, such as a human subject by administration of one or more of the antigenically-cloaked gp120 antigens disclosed herein. Although particular methods, dosages and modes of administrations are provided, one skilled in the art will appreciate that variations can be made without substantially affecting the treatment.

HIV, such as HIV type 1 (HIV-1) or HIV type 2 (HIV-2), is treated by administering a therapeutically effective amount of a disclosed antigenically-cloaked gp120 antigen that induces an immune response to HIV, for example by inducing an immune response, such as a neutralizing antibody response to a protein present on the surface of HIV, for example a gp120 peptide.

Briefly, the method includes screening subjects to determine if they have HIV, such as HIV-1 or HIV-2. Subjects having HIV are selected for further treatment. In one example, subjects are selected who have increased levels of HIV antibodies in their blood, as detected with an enzyme-linked immunosorbent assay, Western blot, immunofluorescence assay or nucleic acid testing, including viral RNA or proviral DNA amplification methods. In one example, half of the subjects follow the established protocol for treatment of HIV (such as a highly active antiretroviral therapy). The other half follow the established protocol for treatment of HIV (such as treatment with highly active antiretroviral compounds) in combination with administration of the agents including a therapeutically effective amount of a disclosed antigenically-cloaked gp120 antigen that induces an immune response to HIV. In another example, half of the subjects follow the established protocol for treatment of HIV (such as a highly active antiretroviral therapy). The other half of the subjects receive a therapeutically effective amount of a disclosed antigenically-cloaked gp120 antigen that induces an immune response to HIV, such as a neutralizing antibody response.

### Screening Subjects

In particular examples, the subject is first screened to determine if the subject has HIV. Examples of methods that can be used to screen for HIV include measuring a subject's CD4+ T cell count and the level of HIV in serum blood levels.

In some examples, HIV testing consists of initial screening with an enzyme-linked immunosorbent assay (ELISA) to detect antibodies to HIV, such as to HIV-1. Specimens with a nonreactive result from the initial ELISA are considered HIV-negative unless new exposure to an infected partner or partner of unknown HIV status has occurred. Specimens with a reactive ELISA result are retested in duplicate. If the result of either duplicate test is reactive, the specimen is reported as repeatedly reactive and undergoes confirmatory testing with a more specific supplemental test (for example, Western blot or an immunofluorescence assay (IFA)). Specimens that are repeatedly reactive by ELISA and positive by IFA or reactive by Western blot are considered HIV-positive and indicative of HIV infection. Specimens that are repeatedly ELISA-reactive occasionally provide an indeterminate Western blot result, which may be either an incomplete antibody response to HIV in an infected person or nonspecific reactions in an uninfected person. IFA can be used to confirm infection in these ambiguous cases. In some instances, a second specimen will be collected more than a month later and retested for subjects with indeterminate Western blot results. In additional examples, nucleic acid testing (for example, viral RNA or proviral DNA amplification method) can also help diagnosis in certain situations.

The detection of HIV in a subject's blood is indicative that the subject has HIV and is a candidate for receiving the therapeutic compositions disclosed herein. Moreover, detection of a CD4+ T cell count below 350 per microliter, such as 200 cells per microliter, is also indicative that the subject is likely to have HIV.

Pre-screening is not required prior to administration of the therapeutic compositions disclosed herein.

### Pre-Treatment of Subjects

In particular examples, the subject is treated prior to diagnosis of AIDS with the administration of a therapeutically effective amount of a disclosed antigenically-cloaked gp120 antigen that induces an immune response to HIV. In some examples, the subject is treated with an established protocol for treatment of AIDS (such as a highly active antiretroviral therapy) prior to treatment with the administration of a therapeutic agent that includes one or more of the disclosed antigenically-cloaked gp120 antigens that induces an immune response to HIV. However, such pre-treatment is not always required and can be determined by a skilled clinician.

### Administration of Therapeutic Compositions

Following selection, a therapeutic effective dose of the a therapeutically effective amount of a disclosed antigenically-cloaked gp120 antigen that induces an immune response to HIV is administered to the subject (such as an adult human or a newborn infant either at risk for contracting HIV or known to be infected with HIV). Additional agents, such as anti-viral agents, can also be administered to the subject simultaneously or prior to or following administration of the disclosed agents. Administration can be achieved by any method known in the art, such as oral administration, inhalation, intravenous, intramuscular, intraperitoneal or subcutaneous.

The amount of the composition administered to prevent, reduce, inhibit, and/or treat HIV or a condition associated with it depends on the subject being treated, the severity of the disorder and the manner of administration of the therapeutic composition. Ideally, a therapeutically effective amount of an agent is the amount sufficient to prevent, reduce, and/or inhibit, and/or treat the condition (for example, HIV) in a subject without causing a substantial cytotoxic effect in the subject. An effective amount can be readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a daily, weekly or monthly repeated administration protocol). In one example, a therapeutically effective amount of a disclosed antigen that induces an immune response to HIV is administered intravenously to a human. As such, these compositions may be formulated with an inert diluent or with a pharmaceutically acceptable carrier. Therapeutic compositions can be taken long term (for example over a period of months or years).

### Assessment

Following the administration of one or more therapies, subjects having HIV (for example, HIV-1 or HIV-2) can be monitored for reductions in HIV levels, increases in a subjects CD4+ T cell count or reductions in one or more clinical symptoms associated with HIV infection. In particular examples, subjects are analyzed one or more times, starting 7 days following treatment. Subjects can be monitored using any method known in the art. For example, biological samples from the subject, including blood, can be obtained and alterations in HIV or CD4+ T cell levels evaluated.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50% or at least 70% reduction of HIV viral load, HIV replication or combination thereof. A partial response may also be an increase in CD4+ T cell count such as at least 350 T cells per microliter.

### Example 4

### Treatment of Subjects

This example describes methods that can be used to treat a subject that has or is at risk of having an infection from a pathogen of interest (such as the pathogens listed in the summary of terms) that can be treated by eliciting an immune response, such as a neutralizing antibody response to the pathogen of interest. In particular examples, the method includes screening a subject having, thought to have or at risk of having a pathogenic infection. Subjects of an unknown infection status can be examined to determine if they have an infection, for example using serological tests, physical examination, enzyme-linked immunosorbent assay (ELISA), radiological screening or other diagnostic technique know to those of skill in the art. In some examples, subjects are screened to identify a particular pathogen of interest, with a serological test, or with a nucleic acid probe specific for a pathogen of interest, or even a panel of nucleic acid probes, such as an array, that can identify several pathogens simultaneously. Subjects found to (or known to) have a pathogenic infection from a pathogen of interest can be administered a disclosed antigen that cam elicit an antibody response to the pathogen of interest. Subjects may also be selected who are at risk of developing a pathogenic infection for example, subjects exposed to a known pathogen of interest, the elderly, the immunocompromised and the very young, such as infants.

Subjects selected for treatment can be administered a therapeutic amount of the disclosed antigen. The disclosed antigen can be administered at doses of 1 μg/kg body weight to about 1 mg/kg body weight per dose, such as 1 μg/kg body weight—100 μg/kg body weight per dose, 100 μg/kg body weight—500 μg/kg body weight per dose, or 500 μg/kg body weight—1000 μg/kg body weight per dose. However, the particular dose can be determined by a skilled clinician. The disclosed antigen can be administered in one or several doses, for example continuously, daily, weekly, or monthly. When administered sequentially the time separating the administration of the disclosed antigen can be seconds, minutes, hours, days, or even weeks.

The mode of administration can be any used in the art. The amount of agent administered to the subject can be determined by a clinician, and may depend on the particular subject treated. Specific exemplary amounts are provided herein (but the disclosure is not limited to such doses).

### Example 5

### Identification of Immunogenic Fragments of gp120

This example describes the selection of immunogenic fragments of cloaked gp120 antigens.

A nucleic acid molecule encoding a cloaked gp120 antigen is expressed in a host using standard techniques (see Sambrook et al., Molecular Cloning; A Laboratory Manual, Cold Spring Harbor Press, Cold Spring Harbor, N.Y. 1989). Preferable cloaked gp120 antigen fragment is expressed such that the cloaked gp120 antigen can be isolated or purified in sufficient quantity. The cloaked gp120 antigen a that are expressed are analyzed by various techniques known in the art, such as immunoblot, and ELISA, and for binding to known neutralizing antibodies of HIV for example, the b12 antibody.

To determine the antigenic potential of cloaked gp120 antigen fragments, subjects such as mice, rabbits or other suitable subjects are immunized with cloaked gp120 antigen fragments. Sera from such immunized subjects are tested for antibody activity for example by ELISA with the expressed polypeptide. They are also tested in a CD4 binding assay, for example by qualitative biacore, and the binding of neutralizing antibodies, for example, by using the b12 antibody. Thus antigenic fragments of are selected to archive broadly reactive neutralizing antibody responses.

While this disclosure has been described with an emphasis upon particular embodiments, it will be obvious to those of ordinary skill in the art that variations of the particular embodiments may be used and it is intended that the disclosure may be practiced otherwise than as specifically described herein. Features, characteristics, compounds, chemical moieties or examples described in conjunction with a particular aspect, embodiment or example of the invention are to be understood to be applicable to any other aspect, embodiment or example of the invention. Accordingly, this disclosure includes all modifications encompassed within the spirit and scope of the disclosure as defined by the following claims.

1. An isolated antigen comprising a target epitope, wherein the target epitope is defined by atomic coordinates of those amino acids of the antigen that contact an antibody of interest that specifically binds the antigen,
wherein the antigen has amino acid substitutions at between about 10% and about 100% of surface exposed amino acid residues located exterior of the target epitope as compared to a wild-type antigen,
wherein the antigen has amino acid substitutions at less than 10% of the non-surface exposed amino acid residues as compared to a wild-type antigen,
wherein the amino acid substitutions alter antigenicity of the antigen in vivo as compared to the wild-type antigen but do not introduce additional glycosylation sites as compared to the wild-type antigen, and
wherein the amino acid substitutions do not significantly alter the binding of the antigen to the antibody of interest.

2. The isolated antigen of claim 1, wherein the wild-type antigen is gp120, wherein the target epitope is defined by those amino acids with greater than 40% surface exposure, that have at least one non-hydrogen atom within 8 angstroms of a non-hydrogen atom of the antibody of interest,
wherein the antigen has amino acid substitutions at between about 10% and about 90% of surface exposed amino acid residues located exterior of the target epitope as compared to a wild-type antigen,
wherein the glycosylation sites in the antigen are not substituted,
wherein surface exposed amino acid residues of the antigen that have a non-hydrogen atom within 6.0 angstroms of any heavy atom on a N-acetyl glucosamine (NAG) group attached to a glycosylation site are not substituted.
wherein the antigen has amino acid substitutions at less than 10% of the non-surface exposed amino acid residues as compared to a wild-type antigen,
wherein the amino acid substitutions alter antigenicity of the antigen in vivo as compared to the wild-type antigen, and
wherein the amino acid substitutions do not significantly alter the binding of the antigen to the antibody of interest.

3. The isolated antigen of claim 2, wherein the antigen comprises the amino acid sequence set forth as one of SEQ ID NOs: 1-9 or an immunogenic fragment thereof or wherein the antigen consists of the amino acid sequence set forth as one of SEQ ID NOs: 1-9.

4. (canceled)

5. The isolated antigen of claim 1 wherein the amino acid substitutions result in the antigen not being bound by antibodies in a polyclonal serum that specifically bind surface exposed amino acid residues of the wild-type antigen located exterior of the target epitope.

6. The isolated antigen of claim 1, wherein between 20% and 100% of surface exposed amino acid residues located exterior to the target epitope are substituted as compared to the wild-type antigen.

7. The isolated antigen of claim 1, wherein the antibody of interest binds the antigen and the wild-type antigen with a dissociation constant (Kd) of 100 nM or less.

8. (canceled)

9. The isolated antigen of claim 1, wherein the antigen is glycosylated.

10. The isolated antigen of claim 1, wherein the antigen is a viral antigen, a bacterial antigen, or a fungal antigen.

11. (canceled)

12. The isolated antigen of claim 10, wherein the viral antigen is a human immunodeficiency virus (HIV)-1 antigen.

13. The isolated antigen of claim 12, wherein the HIV-1 antigen comprises gp120 or an immunogenic fragment thereof.

14. The isolated antigen of claim 12, wherein the antibody is 2F5, 2G12, b12, or 4E10.

15. The isolated antigen of claim 13, wherein the antigen is a trimer of gp120.

16. The isolated antigen of claim 13, wherein the antigen further comprises one or more of a foldon domain, a six-histadine residue tag and a transmembrane domain.

17. The isolated antigen of claim 13, wherein the amino acid substitutions comprise substitutions to homologous residues in gp120 from simian immunodeficiency virus (SIV) or HIV-2.

18. The isolated antigen of claim 13, wherein the antigen comprises a gp120 outer domain.

19. An isolated antigen, wherein the antigen is produced by a method comprising:
obtaining the atomic coordinates of a wild-type antigen, wherein a monoclonal antibody specifically binds the wild-type antigen and amino acids of the wild-type antigen that contact the antibody have been identified;
selecting a target epitope comprising the amino acids of the wild-type antigen that contact the monoclonal antibody; and
selectively mutating at least one surface exposed amino acid residue located exterior to the target epitope of the wild-type antigen, and wherein the affinity of the monoclonal antibody for the antigen is not altered.

20. The isolated antigen of claim 19, wherein selecting the target epitope comprises one or more of obtaining atomic coordinates of a complex of the wild-type antigen and the monoclonal antibody, alanine-scanning mutagenesis or hydrogen-deuterium exchange.

21. (canceled)

22. The isolated antigen of claim 19, wherein selectively mutating at least one surface exposed amino acid residue located exterior to the target epitope of the wild-type antigen results in the antigen not being bound by antibodies in a polyclonal serum that specifically bind surface exposed amino acid residues of the wild-type antigen located exterior of the target epitope.

23. The isolated antigen of claim 19, wherein between 10% and 100% of the exposed amino acid residues exterior to the target epitope of the antigen are mutated.

24. (canceled)

25. The isolated antigen of claim 19, wherein the antibody of interest binds both the antigen and the wild-type antigen with a dissociation constant (Kd) of 100 nM or less.

26. (canceled)

27. The isolated antigen of claim 19, wherein the antigen is a viral antigen, a bacterial antigen, or a fungal antigen.

28. (canceled)

29. The isolated antigen of claim 27, wherein the viral antigen is an HIV-1 antigen.

30. The isolated antigen of claim 29, wherein the HIV-1 antigen comprises gp120.

31. The isolated antigen of claim 30, wherein the antibody is 2F5, 2G12, b12, or 4E10.

32. The isolated antigen of claim 30, wherein the antigen is glycosylated and comprises one or more mutations corresponding to:
a) R419N and K421S;
b) I420N and Q422S;
c) Q422N and I424T;
d) I423N and N425T;
e) Q246N;
f) E267N and E269T;
g) K97N and D99T;
h) Q103N and H105S;
i) N94T;
j) Q114N and L116T;
k) G222N and A224T;
l) 1201N and Q203T;
m) P206N and V208T;
n) I423N and N425T;
o) M434N and A436S;
p) Q442N and R444T; or
q) F210N and P212T, in gp120.

33. The isolated antigen of claim 30, wherein the antigen is a trimer of gp120.

34. The isolated antigen of claim 30, wherein the antigen further comprises one or more of a foldon domain, a six-histadine residue tag and a transmembrane domain.

35. The isolated antigen of claim 30, wherein selectively mutating comprises mutation selectively mutating to homologous residues in gp120 from SIV or HIV2.

36. The isolated antigen of claim 30, wherein the antigen comprises the outer domain of a gp120 polypeptide.

37. An isolated nucleic acid molecule encoding the antigen of claim 1.

38. The isolated nucleic acid molecule of claim 37, wherein the nucleotide sequence comprises the nucleic acid sequence set forth as one of SEQ ID NOs: 10-18 or the nucleotide sequence consists of the nucleic acid sequence set forth as one of SEQ ID NOs: 10-18.

39.-42. (canceled)

43. A pharmaceutical composition comprising the isolated antigen of claim 1 or an isolated nucleic acid molecule encoding the isolated antigen and a pharmaceutically acceptable carrier.

44. A method for generating an immune response in a subject, comprising administering to the subject a therapeutically effective amount of the pharmaceutical composition of claim 43, thereby generating the immune response.

45. A method for treating or preventing a human immunodeficiency type 1 (HIV-1) infection in a subject, comprising administering to the subject a therapeutically effective amount of a first isolated antigen, wherein the first isolated antigen comprises the isolated antigen of claim 2 or an isolated nucleic acid molecule encoding the isolated antigen, thereby treating the subject or preventing infection of the subject.

46. The method of claim 45, further comprising administering a therapeutically effective amount of at least one additional isolated antigen of claim 2, wherein the target epitope of the antigen is identical to the first antigen, and wherein the surface exposed amino acid residues located exterior to the target epitope of the first antigen are not identical to the surface exposed amino acid residues located exterior to the target epitope of the at least one additional isolated antigen.

47. The method of claim 45, further comprising administering a therapeutically effective amount of a polypeptide comprising:
a) a monomeric or trimeric gp140 polypeptide;
b) an monomeric or trimeric wild-type gp120 polypeptide;
c) a wild-type outer domain gp120 polypeptide;
d) a nucleic acid molecule expressing the polypeptide of a-c; or
e) any combination of a-d, above.

48. The method of claim 45, further comprising administering to the subject a therapeutically effective amount of an anti-viral agent.

49. A method for detecting or isolating an HIV-I binding antibody in a subject infected with HIV-I comprising:
providing the antigen of claim 2;
contacting the immunogenic composition with an amount of bodily fluid from the subject; and
detecting binding of the HIV-I binding antibody to the antigen, thereby detecting or isolating the HIV-I binding antibody in a subject.

50. (canceled)
